(19) United States
(12) Patent Application Publication
(10) Pub. No.: US 2020/0101056 A1
(43) Pub. Date: Apr. 2, 2020

(54) USE OF ROCK INHIBITORS FOR PROMOTING SINONASAL EPITHELIAL CELL REPAIR

(71) Applicants: FABIANA C.P. VALERA, MONTREAL (CA); MANON RUFFIN, MONTREAL (CA); DAMIEN ADAM, MONTREAL (CA); BADR IBRAHIM, MONTREAL (CA); EMMANUELLE BROCHIERO, MONTREAL (CA); MARTIN Y. DESROSIERS, MONTREAL (CA)

(72) Inventors: FABIANA C.P. VALERA, MONTREAL (CA); MANON RUFFIN, MONTREAL (CA); DAMIEN ADAM, MONTREAL (CA); BADR IBRAHIM, MONTREAL (CA); EMMANUELLE BROCHIERO, MONTREAL (CA); MARTIN Y. DESROSIERS, MONTREAL (CA)

(21) Appl. No.: 16/589,775

(22) Filed: Oct. 1, 2019

Related U.S. Application Data
(60) Provisional application No. 62/739,985, filed on Oct. 2, 2018.

(30) Foreign Application Priority Data
Jun. 17, 2019 (CA) 3046915

(51) Int. Cl. A61K 31/4409; A61K 9/00; A61P 31/04

(52) U.S. Cl. CPC A61K 31/4409; A61K 9/0043; A61K 45/06; A61P 31/04; A61K 9/0024

(57) ABSTRACT

Novel methods and uses for promoting sinonasal epithelial cell repair in a subject are described. These methods and uses are based on the administration of Rho/ROCK signaling pathway inhibitors, such as ROCK inhibitors. Subjects that may benefit from these methods and uses are those suffering from diseases/conditions associated with impaired nasal and paranasal sinus mucosa integrity and/or defective sinonasal epithelial repair and regeneration, such as rhinosinusitis (e.g., chronic rhinosinusitis, CRS). The Rho/ROCK signaling pathway inhibitors may be used alone or in combination with other active agents, such as steroids and/or antibiotics.

### CROSS REFERENCE TO RELATED APPLICATIONS

The present application claims the benefits of U.S. Provisional application No. 62/739,985 filed Oct.2, 2018, and of Canadian application No. 3,046,915 filed Jun. 17, 2019, which are incorporated herein by reference.

### TECHNICAL FIELD

The present disclosure generally relates to sinonasal epithelial cell repair, and more specifically to the treatment of conditions associated with defective sinonasal epithelial repair and regeneration such as chronic rhinosinusitis (CRS).

### BACKGROUND ART

Chronic rhinosinusitis (CRS) is defined as a persistent inflammation of sinonasal mucosa, lasting for more than 12 weeks¹. This disease affects 4.5 to 12% of the population in North American and European Countries², and seriously compromises the quality of life (QoL) indices in affected individuals^{2,3}. Patients with the CRS with nasal polyps (CRSwNP) phenotype have significantly greater impairment of disease-specific QoL than those with CRS without NP⁴. In addition, 40% of patients will present endoscopic recurrence within 6 months after endoscopic sinus surgery (ESS)⁵, with 20% requiring revision surgery within 5 years⁶.

Despite the high personal, social and economic impact of CRSwNP, its physiopathology is still poorly understood. Increasing experimental evidence suggests that the epithelial barrier plays an important role in the disease process^{7,8}. In addition to its role as a physical barrier to pathogens and irritants, epithelial cells are also responsible for initiating and coordinating defensive responses. Upon stimulation, they secrete several cytokines, such as IL-8, IL-33, IL-25 and thymic stromal lymphopoietin (TSLP), which serve to initiate multiple signaling pathways. In light of this, it is currently believed that reduced barrier function following damage to the epithelial lining is a potent contributor to type 2 immunity⁹. Adequate epithelial repair after injury is essential to restore epithelial barrier integrity and to avoid the subepithelial penetration of inhalant particles and deep tissue colonization with pathogenic bacteria¹⁰, thereby perpetuating inflammation and facilitating subsequent infection.

Recent studies have suggested that epithelial repair and regeneration are altered in CRSwNP. Yu et al. has shown impaired basal cell growth and proliferation¹¹, as well as epithelial remodeling (hyperplasia and squamous metaplasia)¹² in CRSwNP.

There is thus a need for the development of novel approaches for promoting repair of the sinonasal mucosa for the treatment of conditions associated with defective sinonasal epithelial repair and regeneration such as CRS.

The present description refers to a number of documents, the content of which is herein incorporated by reference in their entirety.

### SUMMARY OF THE INVENTION

The present disclosure provides the following items 1 to 55:

1. A method for promoting sinonasal epithelial cell repair in a subject in need thereof comprising administering to said subject an effective amount of a Rho/ROCK signaling pathway inhibitor.

2. The method of item 1, wherein the Rho/ROCK signaling pathway inhibitor is a Rho kinase (ROCK) inhibitor.

3. The method of item 2, wherein the ROCK inhibitor is an antibody or an antigen-binding fragment thereof that binds to ROCK1 and/or ROCK2 and blocks its activity.

4. The method of item 2, wherein the ROCK inhibitor is an antisense compound specifically hybridizes with a nucleic acid encoding human ROCK1 and/or human ROCK2.

5. The method of item 2, wherein the ROCK inhibitor is Fasudil (HA-1077), Hydroxyfasudil, Netarsudil, Ripasudil, RKI-1447, GSK429286A, GSK180736A, GSK269962A, Thiazovivin, AT13148, H1152, Glycyl-H1152, TC-S 7001, AS-1892802, HA-1100, OXA-06, SB-772077B, SR-3677, KD025 (SLx-2119), Y-30141, Y-39983, ZINC00881524 or Y-27632, or a pharmaceutically acceptable salt thereof.

6. The method of item 5, wherein the ROCK inhibitor is Y-27632 or a pharmaceutically acceptable salt thereof.

7. The method of item 6, wherein the ROCK inhibitor is a dihydrochloride salt of Y-27632.

8. The method of any one of items 1 to 7, wherein the subject suffers from rhinosinusitis.

9. The method of item 8, wherein the subject suffers from chronic rhinosinusitis (CRS).

10. The method of item 9, wherein the subject suffers from CRS with nasal polyps (CRSwNP).

11. The method of any one of items 8 to 10, wherein the subject underwent endoscopic sinus surgery (ESS) prior to said administering.

12. The method of any one of items 8 to 11, wherein the rhinosinusitis is associated with a bacterial infection.

13. The method of item 12, wherein the bacterial infection comprises Staphylococcus aureus infection.

14. The method of any one of items 1 to 13, wherein the Rho/ROCK signaling pathway inhibitor is formulated in a pharmaceutical composition with at least one pharmaceutically acceptable carrier or excipient.

15. The method of any one of items 1 to 14, wherein the Rho/ROCK signaling pathway inhibitor is administered intranasally.

16. The method of item 15, wherein the Rho/ROCK signaling pathway inhibitor is coated on a nasal or sinus implant.

17. The method of any one of items 1 to 16, wherein the Rho/ROCK signaling pathway inhibitor is administered in combination with at least one additional active agent.

18. The method of item 17, wherein the at least one additional active agent comprises a steroid and/or an antibiotic.

19. Use of a Rho/ROCK signaling pathway inhibitor for promoting sinonasal epithelial cell repair in a subject.

20. Use of a Rho/ROCK signaling pathway inhibitor for the manufacture of a medicament for promoting sinonasal epithelial cell repair in a subject

21. The use of item 19 or 20, wherein the Rho/ROCK signaling pathway inhibitor is a Rho kinase (ROCK) inhibitor.

22. The use of item 21, wherein the ROCK inhibitor is an antibody or an antigen-binding fragment thereof that binds to ROCK1 and/or ROCK2 and blocks its activity.

23. The use of item 21, wherein the ROCK inhibitor is an antisense compound specifically hybridizes with a nucleic acid encoding human ROCK1 and/or human ROCK2.

24. The use of item 21, wherein the ROCK inhibitor is Fasudil (HA-1077), Hydroxyfasudil, Netarsudil, Ripasudil, RKI-1447, GSK429286A, GSK180736A, GSK269962A, Thiazovivin, AT13148, H1152, Glycyl-H1152, TC-S 7001, AS-1892802, HA-1100, OXA-06, SB-772077B, SR-3677, KD025 (SLx-2119), Y-30141, Y-39983, ZINC00881524 or Y-27632, or a pharmaceutically acceptable salt thereof.

25. The use of item 24, wherein the ROCK inhibitor is Y-27632 or a pharmaceutically acceptable salt thereof.

26. The use of item 25, wherein the ROCK inhibitor is a dihydrochloride salt of Y-27632.

27. The use of any one of items 19 to 26, wherein the subject suffers from rhinosinusitis.

28. The use of item 27, wherein the subject suffers from chronic rhinosinusitis (CRS).

29. The use of item 28, wherein the subject suffers from CRS with nasal polyps (CRSwNP).

30. The use of any one of items 27 to 29, wherein the subject underwent endoscopic sinus surgery (ESS) prior to said use.

31. The use of any one of items 27 to 30, wherein the rhinosinusitis is associated with a bacterial infection.

32. The use of item 31, wherein the bacterial infection comprises Staphylococcus aureus infection.

33. The use of any one of items 19 to 32, wherein the Rho/ROCK signaling pathway inhibitor is formulated in a pharmaceutical composition with at least one pharmaceutically acceptable carrier or excipient.

34. The use of any one of items 19 to 33, wherein the Rho/ROCK signaling pathway inhibitor is for intranasal administration.

35. The use of item 34, wherein the Rho/ROCK signaling pathway inhibitor is coated on a nasal or sinus implant.

36. The use of any one of items 19 to 35, wherein the Rho/ROCK signaling pathway inhibitor is used in combination with at least one additional active agent.

37. The use of item 36, wherein the at least one additional active agent comprises a steroid and/or an antibiotic.

38. A Rho/ROCK signaling pathway inhibitor for promoting sinonasal epithelial cell repair in a subject.

39. The Rho/ROCK signaling pathway inhibitor for use according to item 38, wherein the Rho/ROCK signaling pathway inhibitor is a Rho kinase (ROCK) inhibitor.

40. The Rho/ROCK signaling pathway inhibitor for use according to item 39, wherein the ROCK inhibitor is an antibody or an antigen-binding fragment thereof that binds to ROCK1 and/or ROCK2 and blocks its activity.

41. The Rho/ROCK signaling pathway inhibitor for use according to item 39, wherein the ROCK inhibitor is an antisense compound specifically hybridizes with a nucleic acid encoding human ROCK1 and/or human ROCK2.

42. The Rho/ROCK signaling pathway inhibitor for use according to item 39, wherein the ROCK inhibitor is Fasudil (HA-1077), Hydroxyfasudil, Netarsudil, Ripasudil, RKI-1447, GSK429286A, GSK180736A, GSK269962A, Thiazovivin, AT13148, H1152, Glycyl-H1152, TC-S 7001, AS-1892802, HA-1100, OXA-06, SB-772077B, SR-3677, KD025 (SLx-2119), Y-30141, Y-39983, ZINC00881524 or Y-27632, or a pharmaceutically acceptable salt thereof.

43. The Rho/ROCK signaling pathway inhibitor for use according to item 42, wherein the ROCK inhibitor is Y-27632 or a pharmaceutically acceptable salt thereof.

44. The Rho/ROCK signaling pathway inhibitor for use according to item 43, wherein the ROCK inhibitor is a dihydrochloride salt of Y-27632.

45. The Rho/ROCK signaling pathway inhibitor for use according to any one of items 38 to 44, wherein the subject suffers from rhinosinusitis.

46. The Rho/ROCK signaling pathway inhibitor for use according to item 45, wherein the subject suffers from chronic rhinosinusitis (CRS).

47. The Rho/ROCK signaling pathway inhibitor for use according to item 46, wherein the subject suffers from CRS with nasal polyps (CRSwNP).

48. The Rho/ROCK signaling pathway inhibitor for use according to any one of items 38 to 47, wherein the subject underwent endoscopic sinus surgery (ESS) prior to said use.

49. The Rho/ROCK signaling pathway inhibitor for use according to any one of items 45 to 48, wherein the rhinosinusitis is associated with a bacterial infection.

50. The Rho/ROCK signaling pathway inhibitor for use according to item 49, wherein the bacterial infection comprises Staphylococcus aureus infection.

51. The Rho/ROCK signaling pathway inhibitor for use according to any one of items 38 to 50, wherein the Rho/ROCK signaling pathway inhibitor is formulated in a pharmaceutical composition with at least one pharmaceutically acceptable carrier or excipient.

52. The Rho/ROCK signaling pathway inhibitor for use according to any one of items 38 to 51, wherein the Rho/ROCK signaling pathway inhibitor is for intranasal administration. 53. The Rho/ROCK signaling pathway inhibitor for use according to item 52, wherein the Rho/ROCK signaling pathway inhibitor is coated on a nasal or sinus implant.

54. The Rho/ROCK signaling pathway inhibitor for use according to any one of items 38 to 53, wherein the Rho/ROCK signaling pathway inhibitor is used in combination with at least one additional active agent.

55. The Rho/ROCK signaling pathway inhibitor for use according to item 54, wherein the at least one additional active agent comprises a steroid and/or an antibiotic.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A and 1B show that nasal airway epithelial repair rate is poorest in CRSwNP than in control epithelia under baseline conditions. Cell monolayers obtained from pNECs from control subjects or CRSwNP patients were mechanically injured and epithelial repair rates were measured over a 6 h-period. FIG. 1A: Representative pictures of injured epithelia from a control subject (left) and a CRSwNP patient (right) were obtained from inverted microscope (4×). Pictures were taken immediately after the injury has been made (0h, above) and after a 6 h- period of repair (6 h, below). Wounds have been brightened on the pictures and dotted lines have been drawn along the wound edges for better visualization. FIG. 1B: Quantitative analysis of epithelial repair rate, from 15 patients in each group. Samples were derived at the same time and pNECs cultures were processed in parallel in order to compare control and CRSwNP repair rates. Differences between control and CRSwNP repair rates were assessed by one-way ANOVA followed by Bonferroni post-hoc tests. Statistical differences are indicated as p<0.05 (*).;

FIGS. 2A and 2B show that exposure to S. aureus exoproducts decreases nasal epithelial repair rates. pNECs monolayers obtained from control subjects (FIG. 2A) and CRSwNP patients (FIG. 2B) were mechanically injured and treated with LB (left), SA 0.25% (middle) and SA 0.5% (right), and epithelial repair rates were measured over a 6h-period. Upper panels: Representative pictures of injured epithelia from a control subject (FIG. 2A) and a CRSwNP patient (FIG. 2B) were obtained with an inverted microscope (4×). Pictures were taken immediately after injury (0 h, above) and after a 6 h-period of repair (6 h, below). Wounds have been brightened on the pictures and dotted lines have been drawn along the wound edges for better visualization. Quantitative analysis of epithelial repair rates, from 11 control subjects (FIG. 2A) and 12 CRSwNP patients (FIG. 2B), in control condition (LB) and after exposure to SA exoproducts at 0.25% (v/v) and 0.5% (v/v). The values obtained in each condition were compared by repeated measures ANOVA followed by Bonferroni post-hoc tests. Statistical differences are indicated as p<0.05 (*), p<0.01 (**) and p<0.001 (***).*;

FIGS. 3A and 3B show lamellipodial dynamics of control and CRSwNP nasal epithelial cells are affected by exposure to S. aureus exoproducts. pNECs from control subjects (FIG. 3A) or CRSwNP patients (FIG. 3B) were treated with LB at the beginning of the experiment and imaged every minute for 2 hours. Then, S. aureus exoproducts (SA) were added and recording were pursued for the next 4 h (SA(0-2 h) and SA(2-4 h)). Upper panels: representative kymographs taken from time-lapse series of pNECs from a control subject (FIG. 3A) and a patient with CRSwNP (FIG. 3B). Kymographs depict variations in lamellipodial activity along a one-pixel-wide line drawn perpendicularly to the cell membrane in sequential phase-contrast images. Ascending contours show lamellipodial protrusion, whereas descending contours show lamellipodial retraction. Lower panels: Quantitative analysis of lamellipodial dynamics in pNECs from control subjects (FIG. 3A) and

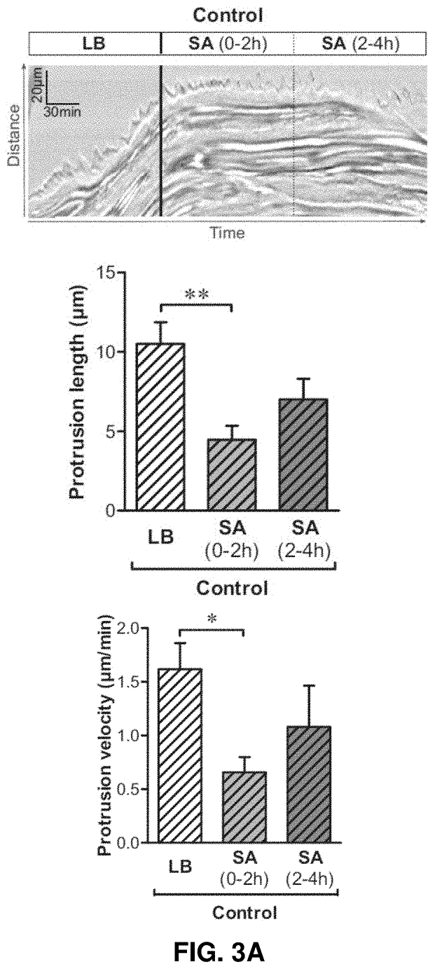

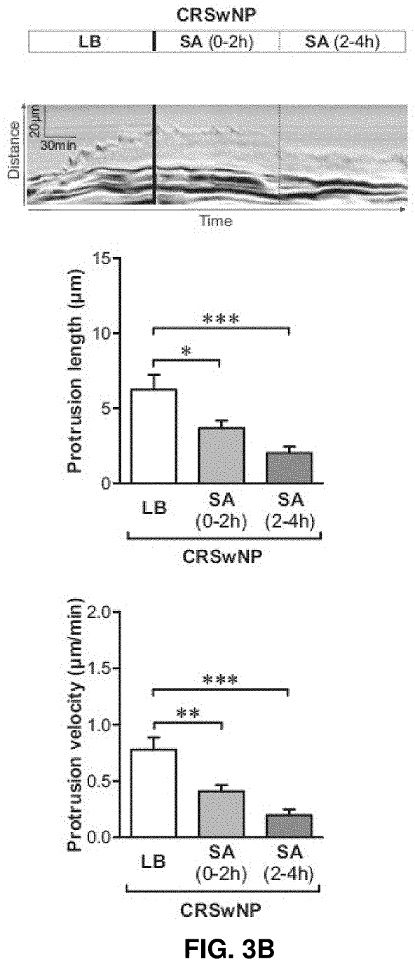

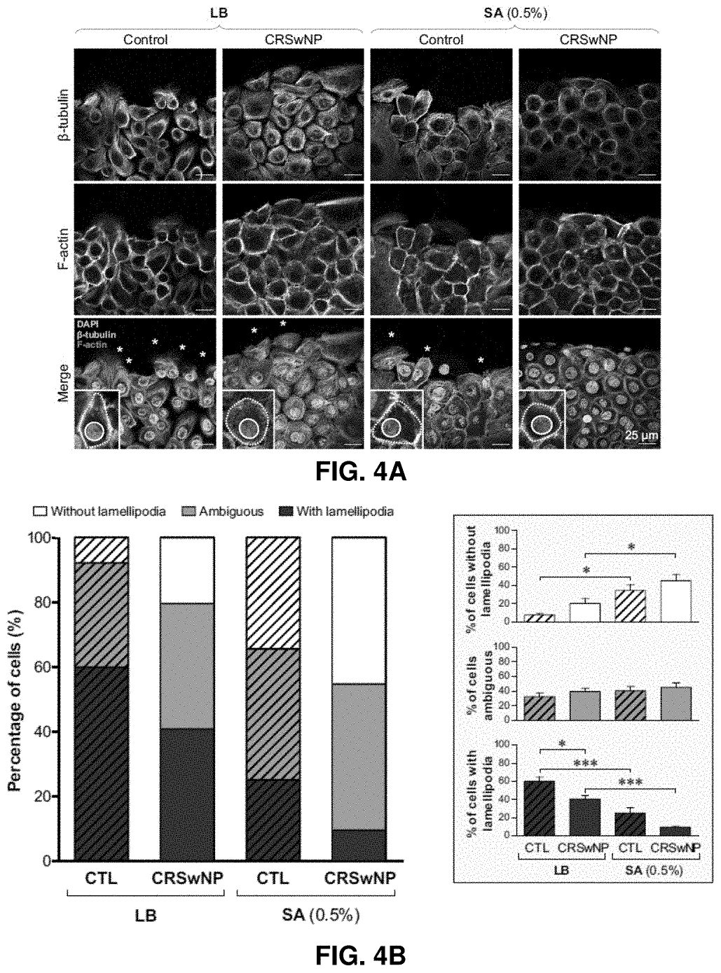

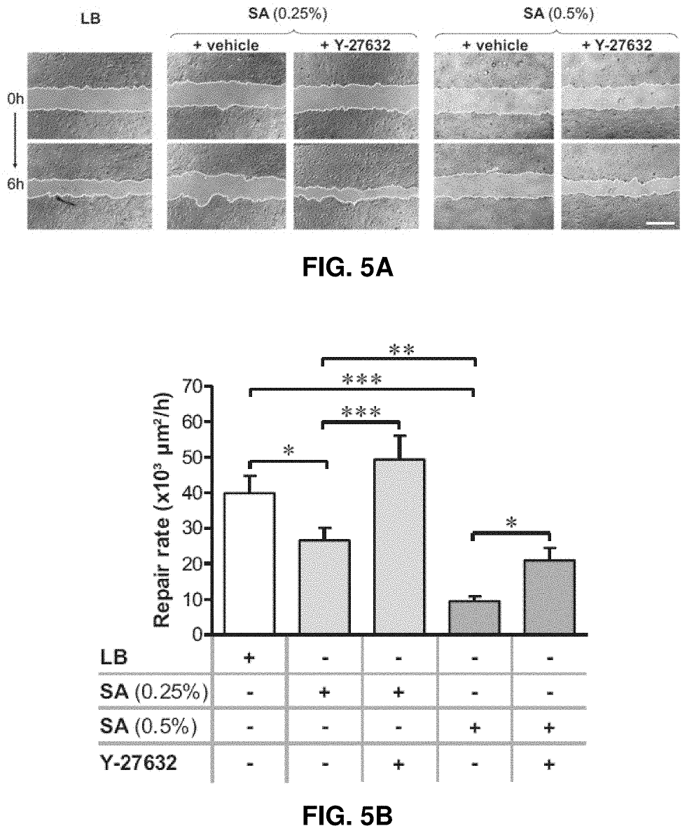

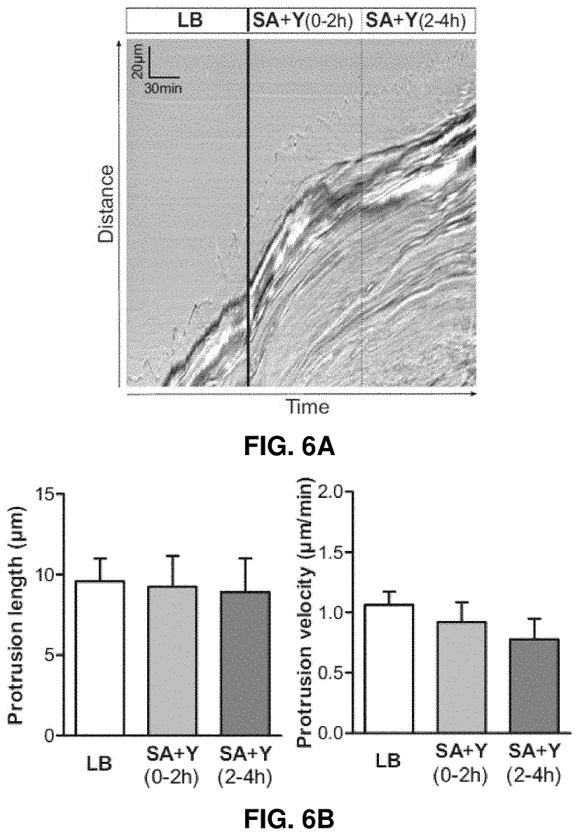

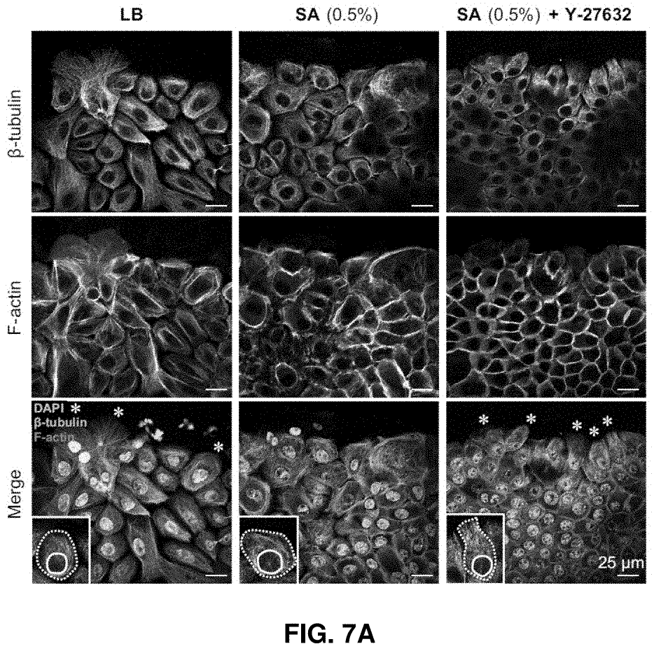

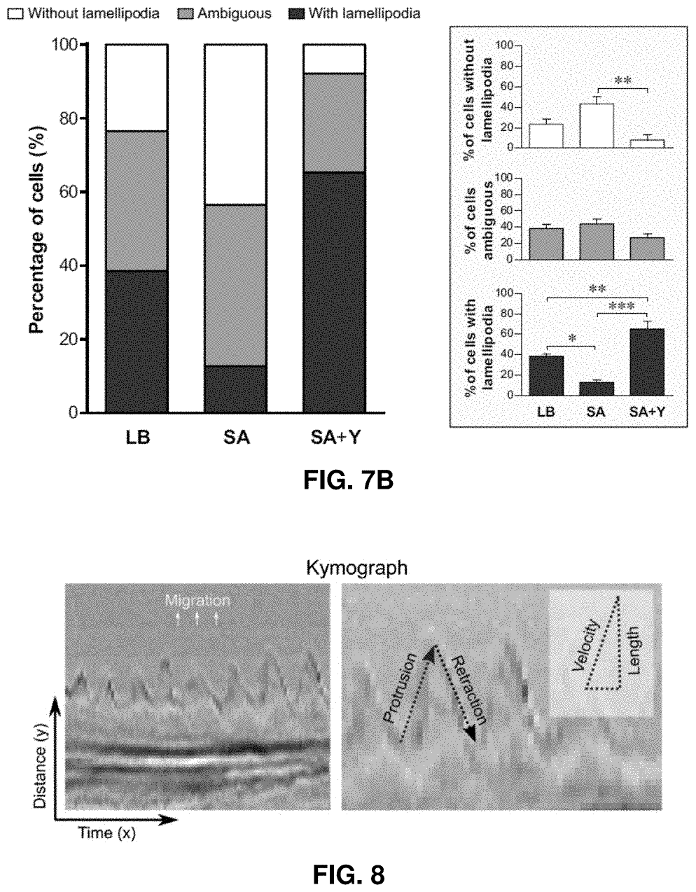

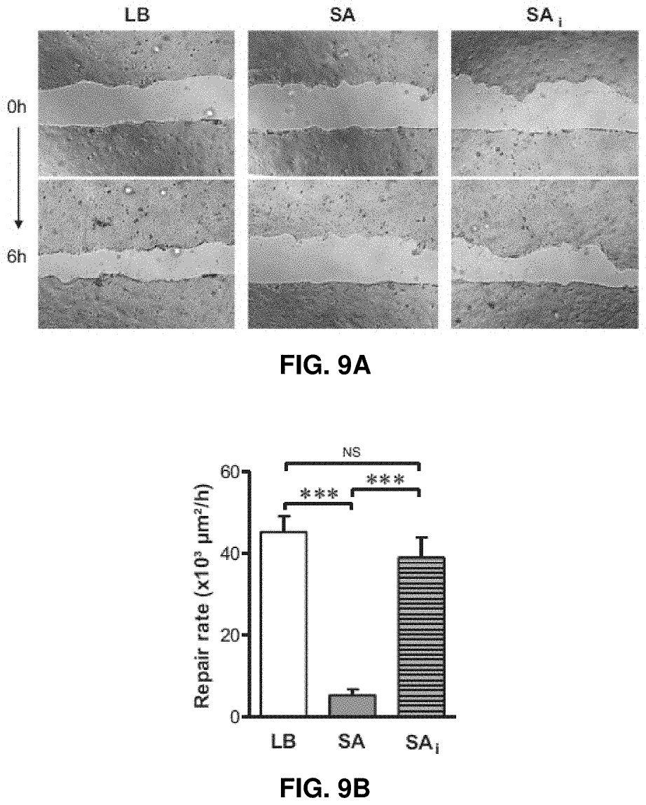

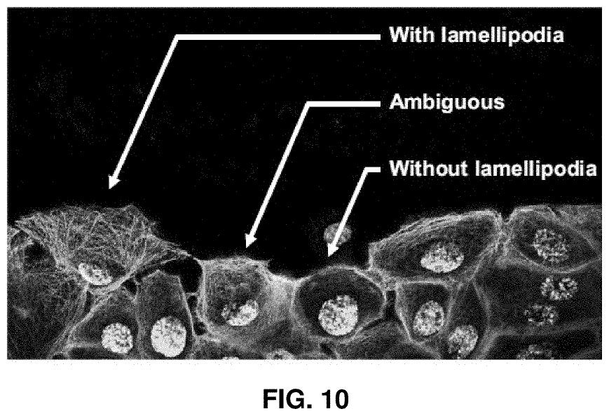

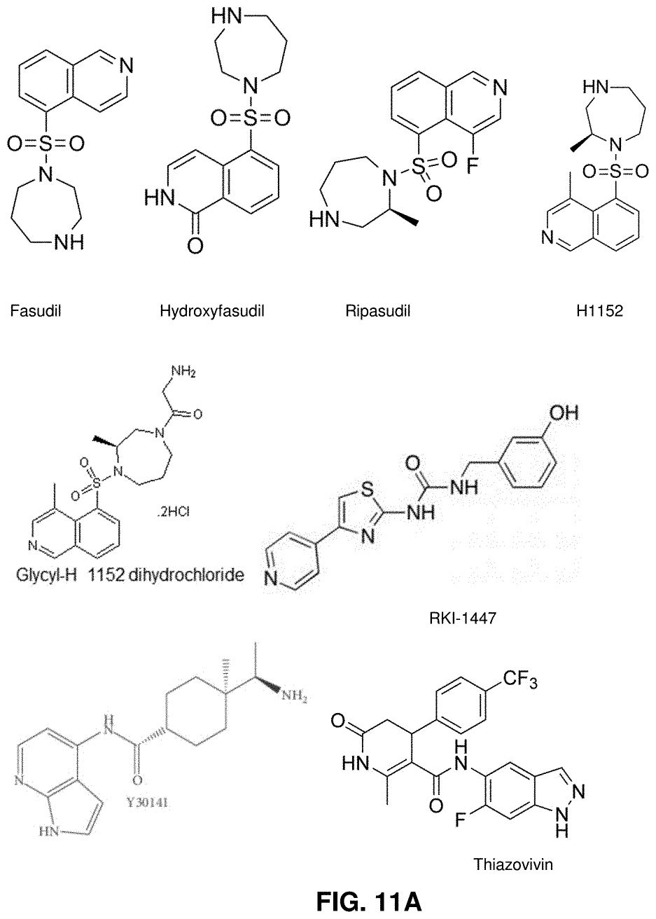

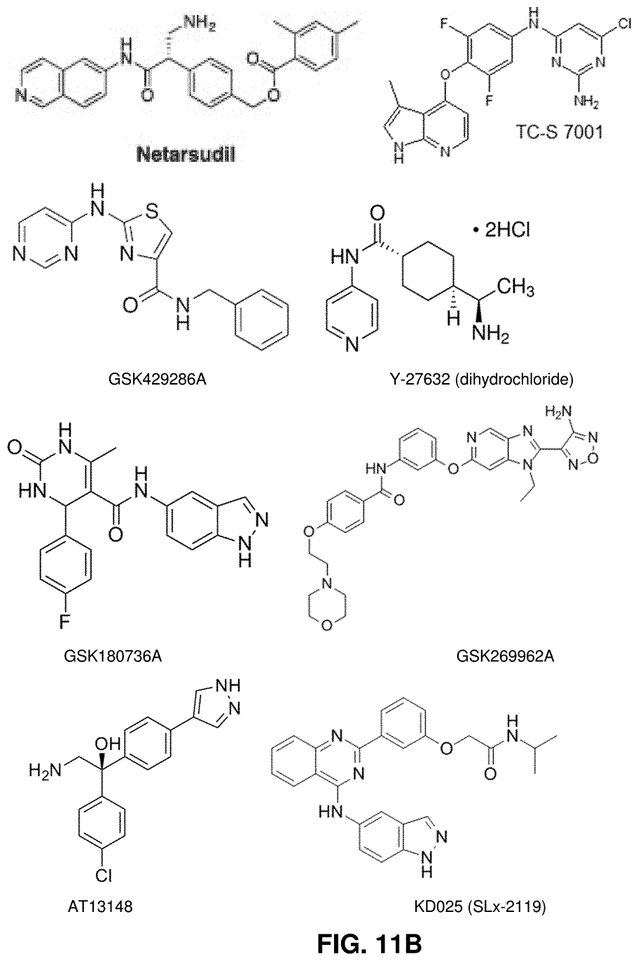

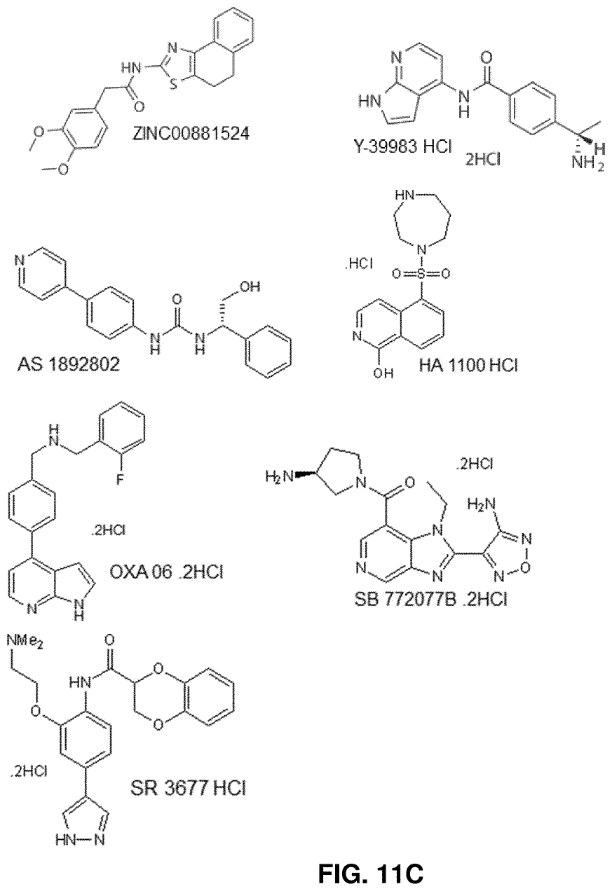

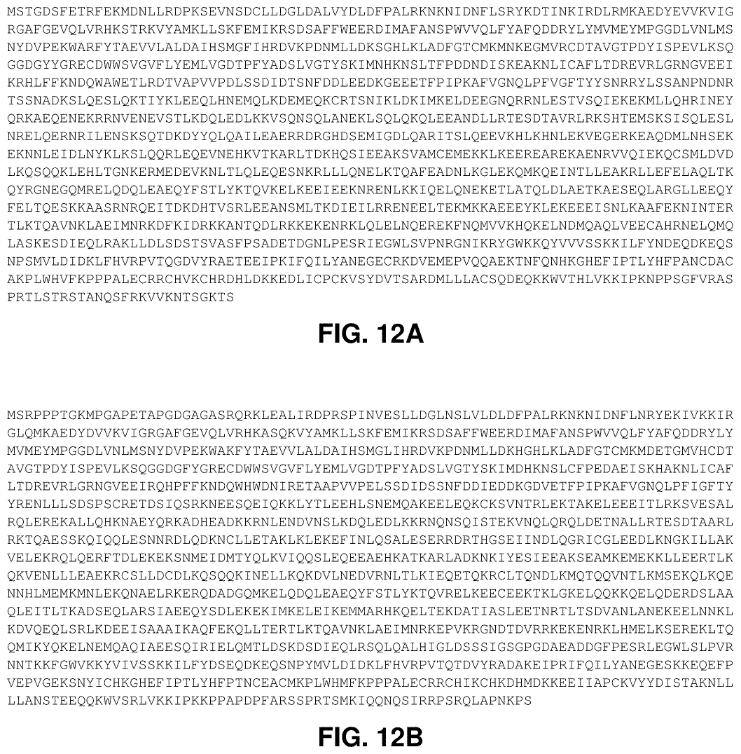

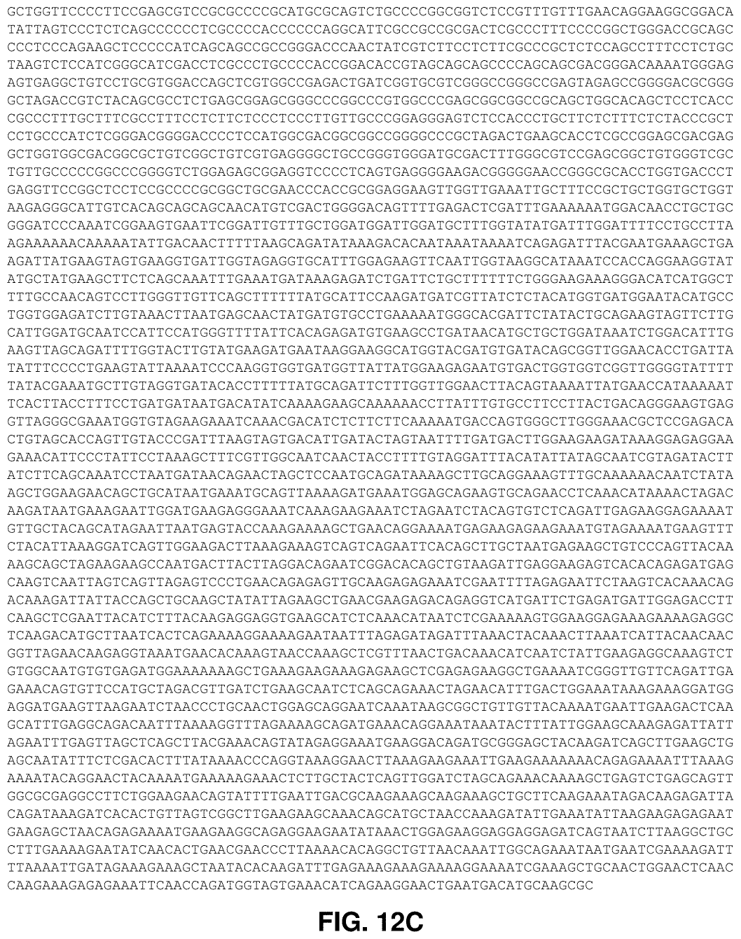

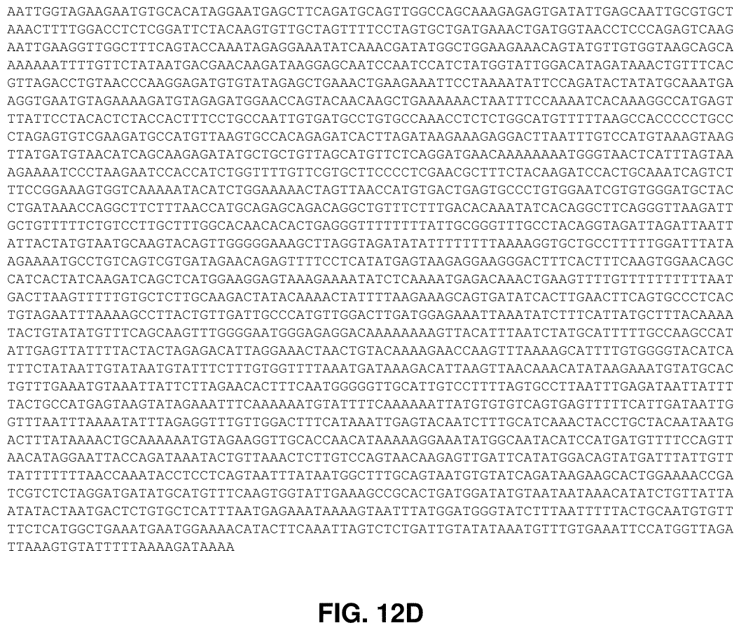

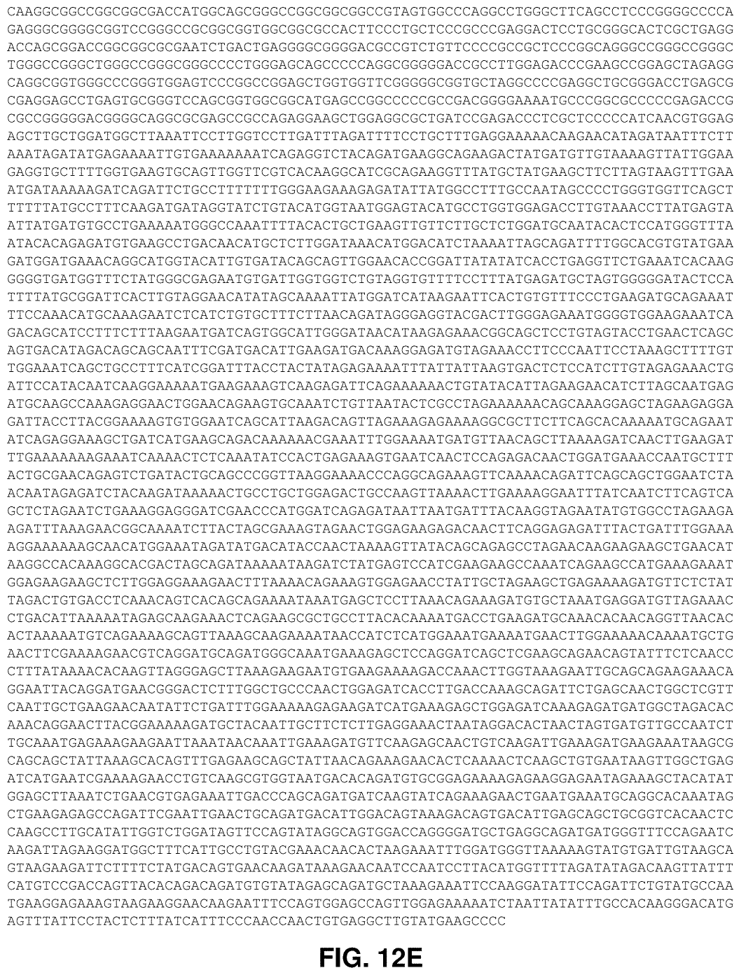

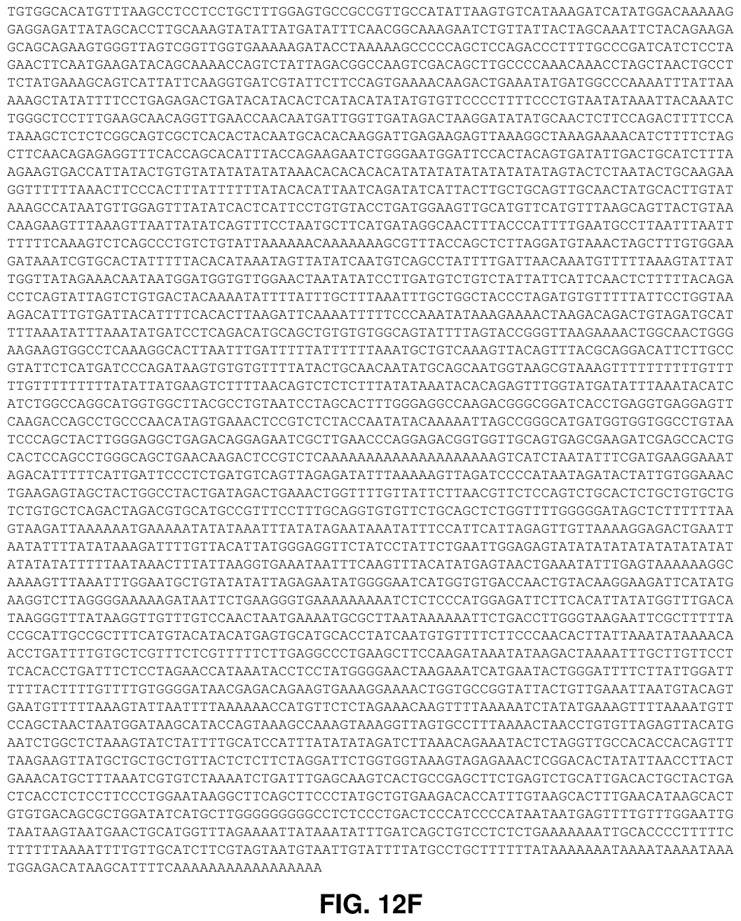

CRSwNP patients (FIG. 3B). Length and velocity of protrusions were calculated from the slopes traced on the leading edge of cells on 14-15 kymographs (each kymograph was taken from one cell) from 3 controls (FIG. 3A) and 3 CRSwNP patients (FIG. 3B). Means were calculated for 3 distinct periods, t=0 h to t=2 h (LB, control condition), t=2 h to t=4 h (SA (0−2 h)) and t=4 h to t=6 h (SA (2−4 h)). Differences between groups (LB, SA (0−2 h) and SA (2−4 h)) were assessed by repeated measures ANOVA followed by Bonferroni post-hoc tests. Statistical differences statistical differences are indicated as p<0.05 (*), p<0.01 (**) and p<0.001 (***).

FIGS. 4A and 4B show that exposure to S. aureus exoproducts affects cytoskeleton organization of nasal epithelia from control subjects and CRSwNP patients. FIG. 4A: Representative confocal microscopy images of nasal epithelia from control subjects and CRSwNP patients mechanically injured and treated with either LB (control condition) or S. aureus exoproducts (SA, 0.5%) for 4 h. Cells were stained for microtubules (with anti-β-tubulin antibody, upper panels) and actin stress fibers (phalloidin staining, middle panels), and nuclei were labeled with DAPI. Cells were visualized using a Leica TCS SP5 confocal microscope (magnification ×63). Merged images are shown on lower panels. White asterisks indicate cell protrusions at the leading edge of the wounds. Insets are shown to emphasize the cell shapes. Scale bars represent 25 μm. If necessary, images were rotated to place the wounds at the top of each photograph. FIG. 4B: Results of the lamellipodial cell quantification. The percentages of cells with lamellipodia (dark grey), with ambiguous morphology (light grey) or without lamellipodia (white) at the wound edge were counted on immunofluorescences images of nasal epithelia from control subjects (Control) and CRSwNP patients (CRS) after mechanical injury and treatment with either LB or SA exoproducts (SA 0.5%). Left panel shows the repartition (in %) of cells according the 3 categories. Values are means ±SEM from n=4 independent experiments (at least 80 cells per condition were classified). Insert (right) shows the results of the statistical analysis. Differences between groups were assessed by one-way ANOVA followed by Bonferroni post-hoc tests. Statistical differences are indicated as follows: p<0.05 (*) and p<0.001 (***).

FIGS. 5A and 5B show that ROCK inhibitor Y-27632 prevented the deleterious effect of S. aureus on epithelial repair rates of CRSwNP nasal epithelial cells. pNECs monolayers from CRSwNP patients were injured, treated with LB+vehicle (water), SA (0.25%)+vehicle (water) and SA (0.25%)+Y-27632, SA (0.5%)+vehicle (water) or SA (0.5%)+Y-27632, and then epithelial repair rates were calculated over a 6h period. FIG. 5A: Representative pictures of injured epithelia from a CRSwNP patient were obtained in the different conditions and taken at 0 h (above) and 6 h (below) after injury with an inverted microscope (4×). Wounds have been brightened on the pictures and dotted lines have been drawn along the wound edges for better visualization. FIG. 5B: Quantitative analysis of epithelial repair rates from 15 CRSwNP patients. The values obtained from each condition within the same group were compared by repeated measures ANOVA followed by Bonferroni post-hoc tests. Statistical differences are indicated as p<0.05 (*), p<0.01 (**) and p<0.001 (***).

FIGS. 6A and 6B show that ROCK inhibition attenuates the negative impact of S. aureus exoproducts on lamellipodial dynamics of nasal epithelial cells from CRSwNP. pNECs monolayers from CRSwNP patients were treated with LB+vehicle (water) at the beginning of the experiment and imaged every minute for 2 h. Then, a combination of 0.5% of SA exoproducts and 5 μM of the ROCK inhibitor Y-27632 (SA+Y) was added, and recording were pursued for the next 4 h. FIG. 6A: Representative kymograph taken from phase-contrast time-lapse series of pNECs from a patient with CRSwNP (right). FIG. 6B: Quantitative analysis of lamellipodial dynamics. Length (left panel) and velocity (right panel) of protrusions were calculated from the slopes traced on the leading edge of cells on 11 kymographs (each kymograph was taken from one cell) from 3 independent experiments (i.e. 3 CRSwNP patients) (N=3 patients, n=11 cells). Means were calculated for 3 distinct periods, t=0 h to t=2 h (LB, control condition), t=2 h to t=4 h (SA+Y (0−2 h)) and t=4 h to t=6h (SA+Y (2−4 h)). Differences between groups were assessed by repeated measures ANOVA followed by Bonferroni post-hoc tests. All comparisons were established as non-significant following statistical analysis (p>0.05).

FIGS. 7A and 7B show that ROCK inhibition protects cytoskeleton disorganization of nasal epithelial cells from CRSwNP due to S. aureus exoproduct exposure. FIG. 7A: Representative confocal microscopy images of nasal epithelia from CRSwNP patients mechanically injured and treated with either LB+vehicle (water, control condition), SA exoproducts (SA 0.5%)+vehicle (water) or SA exoproducts and Y-27632 (SA0.5% +Y-27632 5 μM) for 4 h. Cells were stained for microtubules (with an anti-β-tubulin antibody, upper panels) and actin stress fibers (phalloidin staining, middle panels), and nuclei were labeled with DAPI. Cells were visualized using a Leica TCS SP5 confocal microscope (magnification ×63). Merged images are shown on lower panels. White asterisks indicate protrusions at the leading edge of the wounds. Insets are shown to emphasize the cell shapes. Scale bars represent 25 μm. If necessary, images were rotated to place the wounds at the top of each photograph. FIG. 7B: Results of lamellipodial cell quantification. The percentages of cells with lamellipodia (dark grey), with ambiguous morphology (light grey) or without lamellipodia (white) at the wound edge were counted on immunofluorescences images of nasal epithelia from control subjects (Control) and CRSwNP patients (CRS) after mechanical injury and treatment with either LB+vehicle (LB), SA exoproducts (SA 0.5%)+vehicle (SA) or SA exoproducts (SA 0.5%)+Y-27632 (Y-27632 5 μM) (SA+Y). Left panel shows the repartition (in %) of cells according the 3 categories. Values are means ±SEM from n=4 independent experiments (at least 110 cells per condition were classified). Insert (right) shows the results of the statistical analysis. Differences between groups were assessed by one-way ANOVA followed by Bonferroni post-hoc tests. Statistical differences are indicated as follows: p<0.05 (*) and p<0.001 (***).

FIG. 8 shows a kymograph taken from a phase-contrast time-lapse series showing local protrusion-retraction cycles. Dotted arrows indicate protrusion and retraction phases. Protrusion and retraction length and velocity were calculated using Multiple Kymograph and tsp050706 plugin for ImageJ by tracing a segmented line along the edge of lamellipodia on kymographs.

FIGS. 9A and 9B show that heat-inactivation of SA exoproducts prevents the negative impact of SA exoproducts on the repair rates of CRSwNP nasal epithelia. pNECs monolayers obtained from pNECs from CRSwNP patients were mechanically injured and treated with LB (left), SA exoproducts 0.5% (SA - middle) or heat-inactivated SA exoproducts 0.5% (SA_{inactive}-right) and epithelial repair rates were measured over a 6h-period. Heat inactivation was performed by heating SA exoproducts at 95° C. for 15 minutes. FIG. 9A: Representative pictures were taken immediately after injury (0 h, upper panels) and after 6 h of repair (6 h, lower panels) with an inverted microscope (4×). Wounds have been brightened on the pictures and dotted lines have been drawn at the wound edges for a better visualization. FIG. 9B: Quantitative analysis of epithelial repair rates, from 3 patients. The values obtained from each situation within the same group were compared by repeated measures ANOVA followed by Bonferroni post-hoc tests. Statistical differences are indicated as p<0.001 (***) or non-significant (NS).

FIG. 10 depicts the lamellipodia quantification method used in the studies described herein. Example of cells classified according to the categories with or without lamellipodia as well as with ambiguous morphology. Percentages of cells in each category were then calculated.

FIGS. 11A-11C show the structure of representative ROCK inhibitors.

FIGS. 12A and 12B depict the amino acid sequences of human ROCK1 (UniProtKB-Q13464, SEQ ID NO:2) and ROCK2 (UniProtKB-075116, SEQ ID NO:4), respectively.

FIGS. 12C and 12D depict the nucleotide sequences of the gene encoding human ROCK1 (SEQ ID NO:1).

FIGS. 12E and 12F depict the nucleotide sequences of the gene encoding human ROCK2

(SEQ ID NO:3).

### DISCLOSURE OF INVENTION

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illustrate the invention and does not pose a limitation on the scope of the invention unless otherwise claimed.

No language in the specification should be construed as indicating any non-claimed element as essential to the practice of the invention.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

Unless otherwise defined, all technical and scientific terms used herein have the same meaning as commonly understood by one of ordinary skill in the art to which this invention belongs.

In the studies described herein, the present inventors have shown that there is an intrinsic defect in epithelial repair in CRSwNP patients, and that SA exoproduct exposure alters epithelial repair of normal cells and accentuate the abnormal repair in CRSwNP cells. Furthermore, the studies described herein demonstrate that the effect of SA on cell protrusion is more sustained over time in pNECs cultures from CRSwNP patients. The present inventors have also provided evidence that targeting the Rho/Rho kinase signaling pathway constitutes a new strategy to counteract the negative effect of SA, as the deleterious effect of SA on repair rate and lamellipodia of CRSwNP pNECs cultures was prevented by Rho-associated protein kinase (ROCK) inhibition using the representative ROCK inhibitor Y-27632. These results provide compelling evidence that Rho/ROCK signaling pathway inhibition may be useful for promoting sinonasal epithelial cell repair, and consequently for the treatment of diseases/conditions associated with impairment of nasal and paranasal sinus mucosa integrity and/or sinonasal epithelial repair and regeneration, such as rhinosinusitis.

Accordingly, in a first aspect, the present disclosure provides a method for promoting sinonasal epithelial cell repair in a subject in need thereof comprising administering to said subject an effective amount of a Rho/ROCK signaling pathway inhibitor. The present disclosure also provides the use of a Rho/ROCK signaling pathway inhibitor for promoting sinonasal epithelial cell repair in a subject. The present disclosure also provides the use of a Rho/ROCK signaling pathway inhibitor for the manufacture of a medicament for promoting sinonasal epithelial cell repair in a subject. The present disclosure also provides a Rho/ROCK signaling pathway inhibitor for promoting sinonasal epithelial cell repair in a subject.

In an embodiment, the Rho/ROCK signaling pathway inhibitor is a Rho kinase (ROCK) inhibitor. The term “ROCK inhibitor” as used herein refers to a peptide, a protein, a nucleic acid (antisense oligonucleotides, RNA interference agents such as siRNAs, shRNAs, miRNAs, ribozymes), a small molecule, an antibody or other agent that prevents or reduces expression of ROCK or inhibits ROCK activity, such as its kinase activity.

ROCK is a serine/threonine kinase belonging to the AGC family of protein kinases, which are structurally related to myotonic dystrophy kinase (DMPK) and myotonic dystrophy kinase-related CDC42-binding kinase (MRCK). ROCK is composed of an N-terminal catalytic domain, a central coiled-coil domain, and a C-terminal Pleckstrin homology (PH) domain interrupted by a Cysteine-rich region. ROCK requires both N- and C-terminal extension segments in addition to the core catalytic domain for its activity, which is conserved among the DMPK subgroup. Rho activates ROCK by binding to the C-terminal portion of the coiled-coil. The amino acid sequences of human ROCK1 (UniProtKB-Q13464, SEQ ID NO:2) and ROCK2 (UniProtKB-075116, SEQ ID NO:4) are disclosed in FIG. 12A and FIG. 12B, respectively, and the nucleotides sequences of the cDNAs encoding human ROCK1 and ROCK2 are disclosed in FIGS. 12C-D (SEQ ID NO:1) and FIGS. 12E-F (SEQ ID NO:3), respectively.

In an embodiment, the ROCK inhibitor is an antibody or an antigen-binding fragment thereof that binds to ROCK1 (FIG. 12A) and/or ROCK2 (FIG. 12B) and blocks its activity, for example by blocking its binding to one of its ligand or substrate. The term “antibody or antigen-binding fragment thereof” as used herein refers to any type of antibody/antibody fragment including monoclonal antibodies (including full-length monoclonal antibodies), polyclonal antibodies, multispecific antibodies, humanized antibodies, CDR-grafted antibodies, chimeric antibodies and antibody fragments so long as they exhibit the desired antigenic specificity/binding activity. Antibody fragments comprise a portion of a full-length antibody, generally an antigen binding or variable region thereof. Examples of antibody fragments include Fab, Fab′, F(ab′)_{2,} and Fv fragments, diabodies, linear antibodies, single-chain antibody molecules (e.g., single-chain FV, scFV), single domain antibodies (e.g., from camelids), shark NAR single domain antibodies, and multispecific antibodies formed from antibody fragments. Antibody fragments can also refer to binding moieties comprising CDRs or antigen binding domains including, but not limited to, V_{H} regions (V_{H}, V_{H}-V_{H}), anticalins, PepBodies, antibody-T-cell epitope fusions (Troybodies) or Peptibodies.

In other embodiments, the ROCK inhibitor is an antisense or RNA interference compound. Antisense or RNA interference compounds include, but are not limited to, antisense oligonucleotides, siRNA, miRNA, shRNA and ribozymes. Antisense or RNA interference compounds specifically target ROCK nucleic acids. In one example, a ROCK antisense compound specifically hybridizes with a nucleic acid encoding human ROCK1 (FIGS. 12C-D) and/or human ROCK2 (FIGS. 12E-F).

Several ROCK inhibitors are known in the art and have been described, for example, in the following references: U.S. Pat. Nos. 4,997,834, 4,678,783 and 3,421,217, PCT publication No. WO98/06433, WO99/20620, WO99/61403, WO02/076976, WO02/076977, WO02/100833, WO03/059913, WO03/062227, WO2004/009555, W02004/022541, W02004/108724, W02005/003101, W02005/039564, W02005/034866, WO2005/037197, WO2005/037198,WO2005/035501, WO2005/035503, WO2005/035506, WO2005/080394, WO2005/103050, WO2006/057270, WO2006/058120, WO2007/042321, WO2007/006547, WO2007/026664, WO2008/049919, WO2010/032875, WO2011/107608, WO2011/062765, WO2011/062766, WO2011/130740, WO2012/135697, WO2012/146724, WO2013/030367, WO2013/030216, WO2013/030366, WO2013/112722, WO2014/113620, WO2014/068035, WO2014/118133,

WO2014/134388, WO2014/134391, WO2015/002915, WO2015/002926, WO2015/054317, WO2016/010950, WO2016/028971, WO2016/057306, WO2016/112236, WO2016/144936, WO2017/123860, WO201⁷/₂05709, WO2018/009625, WO2018/009627, WO2018/009622, WO2018/102325, WO/2018/108156, and the like. Such compounds can be produced according to the method described in each of the disclosed references

Examples of representative ROCK inhibitors include Fasudil (HA-1077), Hydroxyfasudil, Netarsudil, Ripasudil, RKI-1447, GSK429286A, GSK180736A, GSK269962A, Thiazovivin, AT13148, H1152, Glycyl-H1152, TC-S 7001, AS-1892802, HA-1100, OXA-06, SB-772077B, SR-3677, KD025 (SLx-2119), Y-30141, Y-39983, ZINC00881524 or Y-27632, or a pharmaceutically acceptable salt thereof, for example a dihydrochloride salt of Y-27632.

In an embodiment, the subject suffers from rhinosinusitis, for example chronic rhinosinusitis (CRS). In an embodiment, the rhinosinusitis is associated with a bacterial infection, e.g., infectious CRS. Bacterial strains associated with rhinosinusitis include Staphylococcus epidermidis, Staphylococcus aureus, Propionibacterium, Streptococcus pneumoniae, Haemophilus influenzae, Pseudomonas aeruginosa, Proteus mirabilis, Klebsiella pneumoniae and Moraxella catarrhalis. In an embodiment, the subject suffers from rhinosinusitis (e.g., CRS) with Staphylococcus aureus infection.

Thus, in another aspect, the present disclosure provides a method for treating rhinosinusitis (e.g., CRS) in a subject in need thereof comprising administering to said subject an effective amount of a Rho/ROCK signaling pathway inhibitor. The present disclosure also provides the use of a Rho/ROCK signaling pathway inhibitor for treating rhinosinusitis (e.g., CRS) in a subject. The present disclosure also provides the use of a Rho/ROCK signaling pathway inhibitor for the manufacture of a medicament for treating rhinosinusitis (e.g., CRS) in a subject. The present disclosure also provides a Rho/ROCK signaling pathway inhibitor for treating rhinosinusitis (e.g., CRS) in a subject.

As used herein, the term “treat”, “treating” or “treatment” is not intended to be absolute terms. Treatment can refer to any delay in onset, reduction in the frequency or severity of symptoms, amelioration of one or more symptoms, improvement in patient comfort and/or respiratory function, etc. The effect of treatment can be compared to an individual or pool of individuals not receiving a given treatment, or to the same patient prior to, or after cessation of, treatment.

In an embodiment, the patient had not undergone sinus surgery prior to said treatment. In another embodiment, the patient had undergone sinus surgery (endoscopic sinus surgery, ESS) prior to said treatment.

In an embodiment, the patient is suffering from steroid-resistant CRS or refractory CRS. The expression “patients suffering from refractory CRS” as used herein refers to patients with CRS refractory to medical and surgical therapy as defined by failure of technically adequate surgery followed by irrigations with a steroid (e.g., a corticosteroid such as budesonide) solution^{1,22} to eliminate the signs and symptoms of CRS. In an embodiment, the patient is non-responsive to a treatment with an anti-inflammatory agent (e.g., steroid), i.e. in whom cessation of anti-inflammatory agent (e.g., steroid) treatment results in no significant deterioration of the signs and/or symptoms of CRS. In another embodiment, the patient is responsive to a treatment with an anti-inflammatory agent (e.g., steroid), i.e. in whom cessation of anti-inflammatory agent (e.g., steroid) treatment results in deterioration of signs and/or symptoms of CRS (i.e., one or more of the symptoms and/or conditions described below). In an embodiment, the subject suffers from CRS with nasal polyps (CRSwNP).

In an embodiment, the treatment comprises improvement of at least one sinus conditions. In another embodiment, the treatment comprises improvement of at least one quality-of-life (QOL) parameters. In another embodiment, the treatment comprises improvement of sinus mucosal aspect. In an embodiment, the treatment comprises improvement of at least one sinus conditions, at least one QOL parameters, and sinus mucosal aspect. In another embodiment, the treatment comprises improvement of at least one of the following CRS symptom parameters: sino-nasal symptom score (SNSS), 22 item Sino-Nasal Outcome Test (SNOT-22®, Washington University School of Medicine, St. Louis, Mo.) score and/or peri-operative sinus endoscopy (POSE) score.

The term “effective amount” refers to that amount of a therapeutic agent (e.g., ROCK inhibitor) sufficient to promote sinonasal epithelial cell repair, e.g., to ameliorate one or more symptoms of rhinosinusitis (e.g., CRS). For example, for a given disease parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

For the prevention, treatment or reduction in the severity of a given disease or condition, the appropriate dosage of the compound/composition will depend on the type of disease or condition to be treated, the severity and course of the disease or condition, whether the compound/composition is administered for preventive or therapeutic purposes, previous therapy, the patient's clinical history and response to the compound/composition, and the discretion of the attending physician. The compound/composition is suitably administered to the patient at one time or over a series of treatments. Preferably, it is desirable to determine the dose-response curve in vitro, and then in useful animal models prior to testing in humans. The present invention provides dosages for the compounds and compositions comprising same. For example, depending on the type and severity of the disease, about 1 μ/kg to to 1000 mg per kg (mg/kg) of body weight per day. Further, the effective dose may be 0.5 mg/kg, 1 mg/kg, 5 mg/kg, 10 mg/kg, 15 mg/kg, 20 mg/kg/ 25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, and may increase by 25 mg/kg increments up to 1000 mg/kg, or may range between any two of the foregoing values. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

In an embodiment, the Rho/ROCK signaling pathway inhibitor is formulated in a pharmaceutical composition comprising at least one pharmaceutically acceptable carrier or excipient.

As used herein, the term “pharmaceutically acceptable” is used synonymously with physiologically acceptable and pharmacologically acceptable with respect to, e.g., a pharmaceutical composition. A pharmaceutical composition will generally comprise agents for buffering and preservation in storage, and can include buffers and carriers for appropriate delivery. Therapeutic formulations are prepared using standard methods known in the art by mixing the active ingredient(s) with one or more optional pharmaceutically acceptable carriers, excipients and/or stabilizers (see, e.g., Remington: The Science and Practice of Pharmacy, by Loyd V Allen, Jr, 2012, 22nd edition, Pharmaceutical Press; Handbook of Pharmaceutical Excipients, by Rowe et al., 2012, 7th edition, Pharmaceutical Press). The composition defined herein may be prepared in any suitable form which does not negatively affect the active agent(s) present in the composition. Selection of the excipients and the most appropriate methods for formulation in view of the particular purpose of the composition is within the scope of ordinary persons skilled in the art of pharmaceutical technology.

In some embodiments, the Rho/ROCK signaling pathway inhibitor or composition comprising same is for sinus administration/delivery. Sinus administration may be performed using an inhalation device, a nasal/sinus implant or stent, a spray, an aerosol, a syringe, nasal drops, nebulization, atomization, by flushing, or by irrigation. In an embodiment, the administration is performed by sinus rinse or irrigation, e.g., irrigation to the nasal and sinus passages. Sinus delivery/administration may be achieved/performed using commercially available kits or systems, e.g., NeilMed SINUS RINSE™ system, hydraSense NetiRinse® Nasal & Sinus Irrigation Kit, SinuPulse™ Elite Advanced Nasal Sinus Irrigation System, Xlear™ Sinus Care Rinse system, Optinose®, or Naväge® irrigator devices. In an embodiment, the Rho/ROCK signaling pathway inhibitor or composition is coated on a nasal/sinus implant or stent, e.g. a drug-eluting nasal/sinus implant or stent (see, e.g., Parikh et al., Pharmaceutics. 2014 Jun; 6(2): 249-267).

In an embodiment, the compositions defined herein further comprise one or more excipients commonly used in nasal formulations (e.g., nasal sprays), such as water, preservatives (e.g., EDTA, benzalkonium chloride, benzethonium chloride, benzyl alcohol, chlorobutanol, methylparaben, phenylethyl alcohol, phenylmercuric acetate, propylene paraben, thimerosal), buffer salts or tonicity agents (e.g., sodium chloride, potassium chloride, glycerol, glycine), viscosity modifying agents (e.g., Me—OH—Pr cellulose, Na-CMC, microcrystalline cellulose), surfactants (glyceryl monoleate, lecithin, polysorbate 20 or 80) suspending agents/solvents (PEG), pH adjusting agents (acetic and citric acids, NaOH, HCl), mucoadhesive polymers (e.g., HPMC, chitosan), fillers/binders (e.g., cellulose-based polymers, maltodextrin, propylene glycol, colloidal silicon dioxide, guar gum) and flavors (e.g., menthol, saccharin sodium, sorbitol).

In an embodiment, the Rho/ROCK signaling pathway inhibitor or composition comprising same is used in combination another therapeutic agent. For example, the Rho/ROCK signaling pathway inhibitor or composition comprising same may be used with agents or therapies used in the treatment of CRS, such as topical intranasal steroids (e.g., prednisone, budenoside), oral antibiotics (e.g., fluoroquinolones, amoxicillin, amoxicillin-clavulanate acid combinations), topical antibiotics, nasal saline irrigation, statins , probiotics, surgery (e.g., endoscopic sinus surgery), oral steroids or combination of oral antibiotics and steroids. The combination of agents may be co-administered (e.g., consecutively, simultaneously, at different times). In an embodiment, the co-administration is coextensive, i.e. occurs during overlapping periods of time. In another embodiment, the Rho/ROCK signaling pathway inhibitor or composition comprising same and the other agent/therapy are administered consecutively. In an embodiment, the Rho/ROCK signaling pathway inhibitor and the other active agent are formulated in the same composition. In another embodiment, the Rho/ROCK signaling pathway inhibitor and the other active agent are formulated in separate compositions. In another embodiment, the Rho/ROCK signaling pathway inhibitor and the other active agent are in different compositions, but these compositions are present together in the same kit or package. The kit or package can optionally include one or more containers, saline solutions (e.g., to reconstitute lyophilized, spray-dried or freeze-dried compositions prior to administration), buffers, written instructions, reference to an internet site, or electronic instructions (e.g. on a support such as a CD-ROM, DVD, USB flash drive), and more particularly instructions for performing the methods and uses described herein, i.e. for treating CRS in a patient.

The subject/patient to be treated may be a child, an adult or an elderly. In an embodiment, the subject/patient is an adult or an elderly.

### MODE(S) FOR CARRYING OUT THE INVENTION

The present invention is illustrated in further details by the following non-limiting examples.

### Example 1: Materials and Methods

A total of 35 samples, composed of 19 cases of CRSwNP and 16 controls were included in this study. The demographic data for each group is presented in Table 1.

TABLE 1
Demographic data of patients whose
samples were used at the present study.
Data are presented by frequency of events.

| Data | CRSwNP | Control |
|---|---|---|
| Age | 51.0 ± 9.5 | 50.3 ± 18.1 |
| Gender | 10F/16M | 8F/8M |
| Previous ESS | 10/26 | — |
| Asthma | 6/26 | 0/16 |
| AERD | 3/26 | 0/16 |
| Smoking | 6/26 | 1/16 |
| IgE ≥ 100 UI/mL | 5/26 | — |

ESS: endoscopic sinus surgery;
AERD: aspirin-exacerbated respiratory disease;
IgE: immunoglobulin E

Surgical sample collection, cell isolation and primary culture.

CRSwNP diagnosis was performed according to the 2004 AAO-HNS guidelines³¹, and patients underwent ESS following failure of medical treatment. Control patients underwent ESS for cerebral spinal fluid leak or non-secreting pituitary adenomas. Exclusion criteria for both groups comprised immune deficiencies, neoplasia, ciliary dyskinesia, cystic fibrosis, and systemic immunosuppression. Tissues obtained during surgery at the level of anterior ethmoidal bulla (control) or the polyp itself (CRSwNP) were immediately collected for subsequent cell isolation and culture. After recovery, pNECs were isolated following a standardized procedure³²⁻³⁵. Briefly, collected samples were immediately rinsed and incubated overnight at 4° C. in MEM (minimum essential medium) medium (Life Technologies Carlsbad, Calif., USA) supplemented with 7.5% NaHCO₃ (Sigma-Aldrich, Saint-Louis, Mo., USA), 2 mM L-glutamine (Life Technologies), 10 mM HEPES (Thermo Fisher Scientific, Waltham, Mass., USA), 0.05 mg/mL gentamycin (Sandoz, Boucherville, QC, Canada), 50 U/mL penicillin-streptomycin, 0.25 μg/mL Fungizone (Life Technologies), 0.1% protease and 10 μg/mL DNase (Sigma-Aldrich).

After 18 h, Fetal Bovine Serum was added to the solution to neutralize the protease-DNase activity, and pNECs were gently scraped off the remaining tissue. Following removal of red blood cells by treatment with ACK lysis buffer (0.1 mM NH₄Cl, 10 μM KHCO₃, 10 nM EDTA), the cell suspension was characterized by immunodetection of specific epithelial markers which indicated a mix of basal (CK13-positive), ciliated (β-tubulin-positive) and goblet (MUCSAC-positive) cells. This freshly isolated pNECs suspension was then seeded in T75 flasks coated with Purecol (Cedarlane Laboratory, Burlington, ON, Canada), and grown in CnT-17 to expend progenitor CK13-positive basal cells, until confluency (CellnTec Advanced Cell Systems, Bern, Switzerland) medium, supplemented with 100U/mL penicillin and streptomycin. pNECs were then detached by trypsin solution; 100 000 cells (second passage) were seeded into 24-well plastic plates (Corning, N.Y., USA), and cultured in CnT-17. At 80% confluency, the CnT-17 medium was replaced by a mix of 1:1 volume of BEGM (bronchial epithelial cell growth medium—Lonza CC4175, Basel, CH) and DMEM (Dulbecco's Modified Eagle's Medium—Thermo Fisher Scientific), supplemented with 1.5 μg/ml BSA, 1×10⁻⁷ M retinoic acid and 100 U/ml of penicillin-streptomycin³²⁻³⁵.

### S. aureus Strains

S. aureus (Rosenbach ATCC®25923 strain, Manassas, Va., USA) was cultured in Luria Bertani (LB, Difco, Montreal, QC, CA) agar, under agitation at 250 rpm at 37° C. Planktonic bacterial cultures at stationary phase after 24 h of growth were centrifuged at 7,200 g for 10 min at room temperature. The supernatant was filtered with 0.22 μm cellulose acetate filters (Corning) to remove bacteria, and filtrates, containing SA exoproducts, were aliquoted and stored at −80° C. One aliquot of this filtrate was heated at 95° C. for 15 minutes to inactivate protease activity (for comparison to non-heat inactivated aliquots). This aliquot was cooled after heating and stored for future assays.

### Wound Healing Assay

pNECs were injured mechanically (3 wounds/well, 3 wells/condition), using a well-established, highly reproducible technique^{32,34,37}. After wounding, the cultures were washed to remove detached cells, and then randomly treated in either of the following conditions (performed in triplicate): 1) negative control: 0.5% (v/v) of LB, 2) exposure to SA exoproducts, (at both concentrations of 0.25% and 0.5% (v/v) SA filtrate), and 3) exposure to SA exoproducts (both concentrations of 0.25% and 0.5% (v/v) SA filtrate) and the ROCK inhibitor Y-27632 dihydrochloride (5 μM, Sigma-Aldrich®).

As a final assay, heat-inactivated-SA exoproducts were used, and the epithelial repair rates were reassessed, at the following conditions: 1) negative control: 0.5% (v/v) of LB, 2) exposure to SA exoproducts (at concentration of 0.5% (v/v) SA filtrate) and 3) exposure to heat-inactivated SA exoproducts (concentration of 0.5% (v/v) SA filtrate).

A mark on wells allowed us to capture images of the wounds at exactly the same place at t=0 h and t=6 h using an inverted microscope with a 4× objective (Nikon Eclipse™ TE 2000). Images were registered using AxioVision™ 4.8 software (Carl Zeiss, Jena, Germany) and then analyzed with Image J software (NIH, Bethesda, Md., USA) to measure areas at t=0 h and t=6 h of wound healing and then to calculate the repair rates (expressed in μm²/h).

### Analysis of Lamellipodial Dynamics by Kymograph

Brightfield time-lapse series were made on pNECs from patients with CRSwNP and control subjects plated in 2-well silicone culture inserts (Ibidi #80209, Fitchburg, USA) using an inverted Axio ObserverZ1 microscope equipped with a CoolSNAP™ EZ CCD camera and a 10×/0.3 NA air objective lens, and then analyzed by AxioVision™ software.

Immediately prior to image acquisition, cells were treated with LB (control condition for SA) or LB+water (control condition for SA+Y-27632). Cells were then positioned into the incubation chamber (37° C., 5% CO₂) and images were captured every minute for 2 h to observe cell protrusions and retractions. Then, cells were treated with either SA exoproducts (concentration of 0.5% (v/v)) or a mix of SA (concentration of 0.5% v/v) and Y-27632 (5 μM), with the recording pursued during the following 4 h.

For each patient, on each time-lapse series, 4 to 6 cells exhibiting lamellipodia with consistent and dynamic protrusion/retraction cycles were randomly chosen for analysis. A one pixel-wide line crossing the cell membrane perpendicularly to the lamellipodia was drawn, and this region was extracted from each image of the time-lapse series, as previously described by Choi et al.³⁸. Kymographs (time-space plots) were generated with the “Multiple Kymograph” plugin (J. Rietdorf, FMI Basel and A. Seitz, EMBL, Heidelberg, Germany) for ImageJ. On kymographs, the x-axis represents time while the y-axis is the distance, where each protrusion/retraction cycle appears as a peak (FIG. 8). On each kymograph, segments following protrusions were manually retraced with the ImageJ segmented line tool. Data derived from these segmented lines were generated using “read velocity tsp macro” of ImageJ and used to calculate the length and velocity of each protrusion. At least a total of 40-110 individual protrusion events per condition and at least 14 different cells were analyzed in 3 independent time-lapse series.

### Immunofluorescence

pNECs were grown to confluency on coverslips (precision cover glasses thickness No. 1.5H, Marienfeld, Lauda-Konigshofen, Germany) coated with Purecol™. After injury, (3 mechanical wounds/coverslip), cell monolayers were washed and then treated with either: 1) LB (concentration of 0.5% (v/v)): negative control, 2) SA exoproducts (concentration of 0.5% (v/v) SA filtrate) and 3) SA exoproducts (concentration of 0.5% (v/v) SA filtrate) and Y-27632 (5 μM). After 4 h of repair, cell monolayers were fixed with 4% paraformaldehyde in PBS for 5 min, permeabilized with 0.1% Triton™ X-100 in PBS for 10 min. After blocking (in 1% BSA in PBS for at least 1h at 4° C.), fixed cells were incubated with the primary anti-β-tubulin antibody (1:1000 in PBS+BSA1%, Millipore, Etobicoke, Canada) for 1 h at room temperature, and then with Alexa Fluor®-488 conjugated goat anti-mouse antibody secondary antibody (1:2000 in PBS+BSA1%,1 h, room temperature Thermo Fischer Scientific, Waltham, USA). F-actin was stained with Phalloidin-Alexa Fluor® 594 (1 unit, Thermo Fischer Scientific) (in PBS+BSA1%, for 1 h at room temperature) and nuclei with DAPI (1:1000 in PBS+BSA1%, for 5 min). Coverslips were mounted with ProLong® Gold Antifade Reagent (Molecular Probes, Eugene, USA) on SuperFrost™ glass slides (Thermo Fisher Scientific). Images were acquired with an inverted confocal microscope (TCS SP5, Leica Microsystems, Wetzlar, Germany) using an oil-immersion objective 63×/1.4. On immunofluorescence images, cells at the wound edge were classified according to the categories “with” or “without” lamellipodia as well as “ambiguous morphology” (see FIG. 10)³⁹. Percentages of cells in each category were then calculated.

### Statistical Analysis

The data from all experiments are presented as mean ±SEM. ANOVA with Bonferroni post-hoc tests were used to compare the different conditions in the same group, and to compare the same condition between groups. Comparisons were considered statistically significant if p<0.05. In figures, statistical differences are indicated as p<0.05 (*), p<0.01 (**) and p<0.001 (***) or non-significant (NS), Graph Pad Prism version 6.0 (GraphPad Software, San Diego, Calif., USA) was used to analyze all results.

Example 2: Effect of SA on Control and CRSwNP Samples

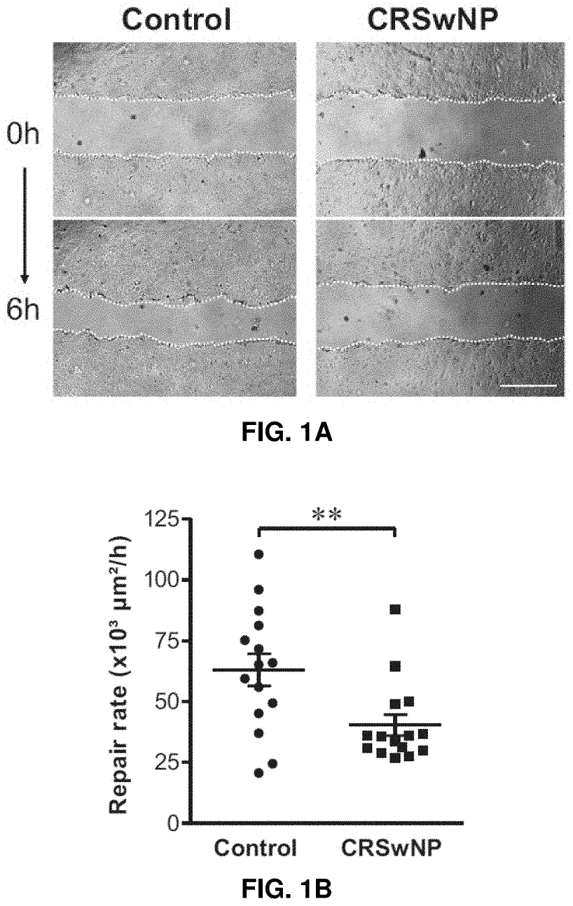

During the last years, sinonasal epithelium has been intrinsically imbricated to CRSwNP physiopathology. Indeed, the epithelial barrier defense is determinant to maintain mucosal homeostasis, either due to its physical (avoiding the entrance of pathogens) and functional implication (defense against pathogens through the release of inflammatory and antimicrobial substances)⁴⁰⁻⁴³. To this end, the epithelium is a key inducer of both innate and adaptive inflammation⁴⁰⁻⁴³. Very recently, Schleimer⁴³ proposed that mucosal injury and repair may be a key process at CRSwNP: a prolonged loss of barrier function of the epithelium may facilitate the entrance of pathogens/inhalants, and thus facilitate chronic inflammation. It was thus evaluated whether the ability of nasal epithelia to repair differed between CRSwNP and control samples under baseline culture conditions. For this purpose, pNECs from controls and CRSwNP were cultured in parallel, in the same conditions, and then wounded, to compare their repair rates. The 2D cultures are predominantly composed of basal progenitor cells, playing a critical role in epithelial repair through their ability to migrate into the injured area and then proliferate^{11,44}. Repair rate analysis (FIGS. 1A, 1 B) in each pNECs cultures from control subjects and CRSwNP patients (CRSwNP) revealed a variability among individual within each group. However, the mean wound repair rates were significantly slower (p<0.001) in pNECs monolayers from CRSwNP than controls, under baseline conditions (LB) (FIG. 1 B).

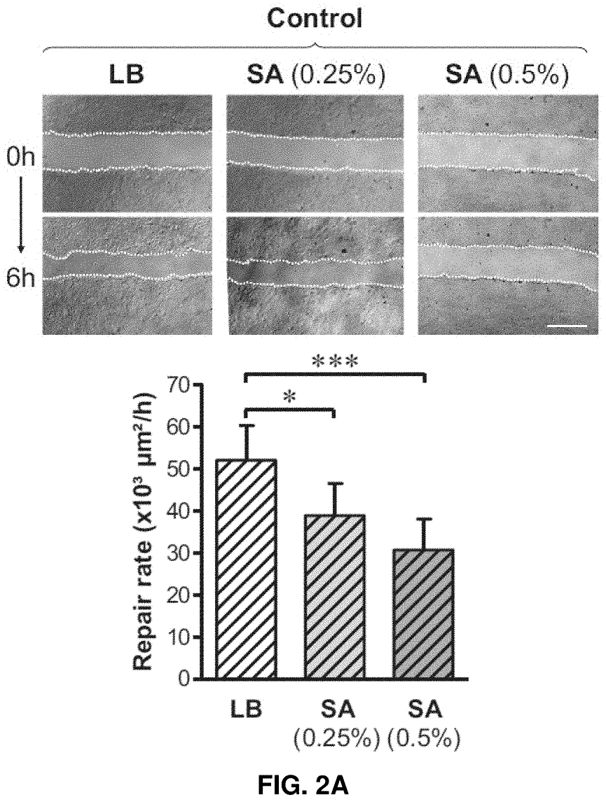

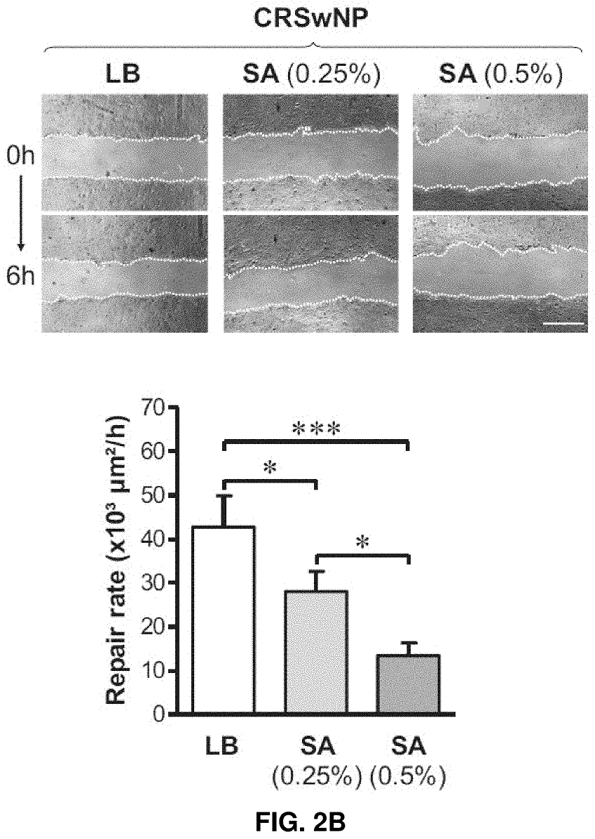

It was then assessed whether exposure to SA exoproducts could alter the wound healing capacity of pNECs epithelia. A significant decrease in the repair of control pNECs monolayers was observed in the presence of 0.25% SA exoproducts (FIG. 2A). A stronger inhibitory effect (p<0.001) was observed after exposure to SA exoproducts at 0.5% (41% decrease compare to the repair rates in LB condition). A dose dependent inhibition of wound repair was observed in CRSwNP monolayers (34% and 69% decrease in the presence of SA 0.25% (p<0.01 vs. LB) and SA 0.5% (p<0.01 vs. LB), respectively (FIG. 2B).

To determine which component could be responsible for this deleterious response, heat-inactivated SA 0.5% exoproducts (SA_{inactive}) were used, and their effect on the epithelial repair of pNECs monolayers from CRSwNP patients was reassessed (FIG. 9). It was observed that the epithelial repair was only weakly influenced by heat-inactivated SA 0.5% exoproducts (SA_{inactive} vs. LB: 14% decrease, non-significant), in contrast to active SA 0.5% exoproducts within the same pNECs cultures (LB vs. SA: 88% decrease, p<0.001; and SA vs. SAᵢ: 86% increase, p<0.001).

Epithelial repair is dependent on several mechanisms, one of them being the coordinated and directional cell migration⁴⁵. To optimize this lively process, the actin filaments organize themselves in a dynamic network, and create a sheet-like membrane protrusion, known as lamellipodia⁴⁵⁻⁴⁷. Lamellipodia formation and orientation was thus further evaluated using video kymograph analysis. It was first confirmed that LB per se had no effect on cell movement.

Kymographs of pNECs cultures for both controls and CRSwNP before and after exposure to SA exoproducts were subsequently evaluated.

Control cells depicted a mean protrusion length and velocity of 10.5±1.36 μm and 1.62±0.24 μm/min, respectively, in LB conditions. Subsequent exposure to SA exoproducts (SA 0-2h) induced a 57% (p<0.01) and 60% (p<0.05) decrease in protrusion length and velocity, respectively, followed by a partial recovery of these parameters during the final two hours of the study observation (SA 2-4h) (FIG. 3A, 3B). In CRSwNP cells, both protrusion length (6.23±0.99 pm) and velocity (0.78±0.11 pm/min) were altered at baseline (LB condition) and the exposure to SA exoproducts significantly worsened the levels of protrusion length and velocity. Moreover, in contrast with control pNECs, the effect of SA was persistent throughout the entire period following exposure to SA exoproducts (SA 0-2 h and SA 2-4 h).

Finally, the cellular cytoskeleton organization was evaluated by immunofluorescence experiments. Microtubules and actin microfilaments were stained with a specific β-tubulin antibody and with fluorescent phalloidin, respectively. Under LB condition, it was observed that the leading cells at the wound edge of injured pNECs monolayers from control subjects exhibited more pronounced lamellipodial extensions (see cells marked with an asterisk, FIG. 4A, merge) than the pNECs cultured from CRSwNP samples. However, control and CRSwNP pNECs appeared to be correctly polarized and oriented towards the injured area (FIG. 4A). There was no substantial difference in β-actin or 3-tubulin distribution within the control and CRSwNP pNECs. However, further analysis and lamellipodial cell quantification at the wound edge showed distinct patterns for control and CRSwNP pNECs (FIG. 4B). Indeed, the percentage of cells with lamellipodia was significantly lower in epithelia from CRSwNP patients than in those from control subjects (Control: 60% vs. CRSwNP: 41%), whereas the percentage of cells without lamellipodia was higher (Control: 8% vs. CRSwNP: 20%) (FIG. 4B).

Both control and CRSwNP pNECs were altered after exposure to SA exoproducts, exhibiting a more rounded shape, suggesting a loss in cell polarization, and a reorganization of the microtubule cytoskeleton, with an increase in microtubules localized circularly under the plasma membrane (FIG. 4A). Additionally, lamellipodial cell quantification showed that SA exoproduct exposure induced significant decreases in the percentage of cells with lamellipodia at the wound edge (Control subjects: LB: 60% vs. SA: 25%; CRSwNP patients: LB: 41% vs. SA: 10%) (FIG. 4B). Conversely, the percentage of cells without lamellipodia is significantly increased after SA exoproduct exposure in epithelia from control subjects (LB: 8% vs. SA: 34%) and CRSwNP patients (LB: 20% vs. SA: 45%) (FIG. 4B).

### Example 3: Effect of ROCK Inhibition on Epithelial Repair in pNECs

The effect of a potent Rho kinase (ROCK) inhibitor, Y-27632, on epithelial repair in pNECs exposed to SA exoproducts was assessed. It was first determined, in a pilot study, that 5 μM was the optimal dose for Y-27632 in pNEC. Indeed, treatment with 5 μM of Y-27632, in LB condition, led to an increase in epithelial repair rate of 23%, while increasing concentrations altered migration and induced pNECs apoptosis

pNECs monolayers were then exposed to SA exoproducts either in the presence or absence of Y-27632 (5 μM) (FIG. 5). The drug inhibited the deleterious effect of SA, increasing significantly the repair rates by almost 2-fold (1.86-fold) when co-cultured with SA 0.25%, and by 2.22-fold with SA 0.5%, compared to corresponding SA conditions in the absence of Y-27632.

It was also assessed whether the beneficial effect of Y-27632 on epithelial repair in the presence of SA exoproducts was related to an improvement in lamellipodial dynamics and migration. Whereas both protrusion length and velocity in CRSwNP pNECs were altered by the exposure to SA exoproducts (FIG. 3), this deleterious effect was no longer observed (FIG. 6) when CRSwNP pNECs were exposed simultaneously to SA 0.5% and Y-27632. Indeed, the length and the velocity of protrusions measured after treatments with SA+Y were similar to those observed in the absence of SA (FIG. 6, LB vs. SA+Y). Finally, the cellular cytoskeleton organization after exposure to SA, in the absence and presence of Y-27632, was compared. It was first confirmed that, in the absence of Y-27632, SA exposure induced the cells to adopt a rounded shape, with lamellipodial extension substantially compromised at the leading edge of injury (FIG. 7A). Treatment with Y-27632 partially restored lamellipodial extensions: although the extension was not as prominent as under baseline conditions, most of the cells at the leading edge presented a more elongated shape and exhibited the lamellipodia pattern directed towards the injured area (FIG. 7A, see cells marked with an asterisk at the leading edge in LB and SA+Y conditions). Interestingly, lamellipodial cell quantification showed that, after exposure to SA exoproduct combined with the Y-27632 compound, injured epithelia from CRSwNP patients exhibited significantly more cells with lamellipodia (SA: 13% vs. SA+Y: 65%) and significantly less cells without lamellipodia (SA: 43% vs. SA+Y: 8%) (FIG. 7B), confirming that ROCK inhibition improves lamellipodial dynamics and migration.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Orlandi R R, Kingdom T T, Hwang P H, Smith T L, Alt J A, Baroody F M, et al. International Consensus Statement on Allergy and Rhinology: Rhinosinusitis. Int Forum Allergy Rhinol. 2016;6:S22-209.
2. DeConde A S, Soler Z M. Chronic rhinosinusitis: Epidemiology and burden of disease. Am J Rhinol Allergy. 2016;30:134-9.
3. Soler Z M, Wittenberg E, Schlosser R J, MacE J C, Smith T L. Health state utility values in patients undergoing endoscopic sinus surgery. Laryngoscope. 2011;121:2672-8.
4. Erskine S, Hopkins C, Kumar N, Wilson J, Clark A, Robertson A, et al. A cross sectional analysis of a case-control study about quality of life in CRS in the UK; a comparison between CRS subtypes. Rhinology. 2016;54:311-5.
5. DeConde A S, Mace J C, Levy J M, Rudmik L, Alt J A, Smith T L. Prevalence of polyp recurrence after endoscopic sinus surgery for chronic rhinosinusitis with nasal polyposis. Laryngoscope. 2017;127(3):550-5.
6. Hopkins C, Slack R, Lund V, Brown P, Copley L, Browne J. Long-term outcomes from the english national comparative audit of surgery for nasal polyposis and chronic rhinosinusitis. Laryngoscope. 2009;119:2459-65.
7. Soyka M B, Wawrzyniak P, Eiwegger T, Holzmann D, Treis A, Wanke K, et al. Defective epithelial barrier in chronic rhinosinusitis: The regulation of tight junctions by IFN-γ and IL-4. J Allergy Clin Immunol. 2012;130:1087-96.
8. Pothoven K L, Norton J E, Hulse K E, Suh L A, Carter R G, Rocci E, et al. Oncostatin M promotes mucosal epithelial barrier dysfunction, and its expression is increased in patients with eosinophilic mucosal disease. J Allergy Clin Immunol. 2015; 136:737-46.
9. Loxham M, Davies D E. Phenotypic and genetic aspects of epithelial barrier function in asthmatic patients. J Allergy Clin Immunol. 2017;139:1736-51.
10. Georas S N, Rezaee F. Epithelial barrier function: at the front line of asthma immunology and allergic airway inflammation. J Allergy Clin Immunol. 2014;134:509-20.
11. Yu X M, Li C W, Chao S S, Li Y Y, Yan Y, Zhao X N, et al. Reduced growth and proliferation dynamics of nasal epithelial stem/progenitor cells in nasal polyps in vitro. Sci Rep. 2014;4:4619.
12. Yu X M, Li C W, Li Y Y, Liu J, Lin Z Bin, Li T Y, et al. Down-regulation of EMP1 is associated with epithelial hyperplasia and metaplasia in nasal polyps. Histopathology. 2013;63:686-95.
13. Bosse Y, Bacot F, Montpetit A, Rung J, Qu H Q, Engert J C, et al. Identification of susceptibility genes for complex diseases using pooling-based genome-wide association scans. Hum Genet.
14. Thanasumpun T, Batra P S. Endoscopically-derived bacterial cultures in chronic rhinosinusitis: A systematic review. Am J Otolaryngol - Head Neck Med Surg. 2015;36:686-91.
15. Zhang Z, Adappa N D, Doghramji L J, Chiu A G, Cohen N A, Palmer J N. Different clinical factors associated with Staphylococcus aureus and Pseudomonas aeruginosa in chronic rhinosinusitis. Int Forum Allergy Rhinol. 2015;5:724-33.
16. Corriveau M N, Zhang N, Holtappels G, Van Roy N, Bachert C. Detection of Staphylococcus aureus in nasal tissue with peptide nucleic acid-fluorescence in situ hybridization. Am J Rhinol Allergy. 2009;23:461-5.
17. Tan N C W, Foreman A, Jardeleza C, Douglas R, Vreugde S, Wormald P J. Intracellular Staphylococcus aureus: The Trojan horse of recalcitrant chronic rhinosinusitis? Int Forum Allergy Rhinol. 2013;3:261-6.
18. Kim R J T, Yin T, Chen C J J, Mansell C J, Wood A, Dunbar P R, et al. The interaction between bacteria and mucosal immunity in chronic rhinosinusitis: A prospective cross-sectional analysis. Am J Rhinol Allergy. 2013;27:183-90.
19. Tan N C W, Cooksley C M, Roscioli E, Drilling A J, Douglas R, Wormald P J, et al. Small-colony variants and phenotype switching of intracellular Staphylococcus aureus in chronic rhinosinusitis. Allergy. 2014;69:1364-71.
20. Kohanski M A, Lane A P. Sinonasal epithelial cell response to Staphylococcus aureus burden in chronic rhinosinusitis. JAMA Otolaryngol Head Neck Surg. 2015;141:341-9.
21. Gevaert E, Zhang N, Krysko O, Lan F, Holtappels G E, De Ruyck N, et al. Extracellular eosinophilic traps in association with Staphylococcus aureus at the site of epithelial barrier defects in patients with severe airway inflammation. J Allergy Clin Immunol. 2017;139:1849-60.
22. Teufelberger A R, Nordengrun M, Braun H, Maes T, De Grove K, Holtappels G, et al. The IL-33/ST2 axis is crucial in type 2 airway responses induced by the Staphylococcus aureus protease SpID. J Allergy Clin Immunol. 2017; Epub Ahead.
23. Malik Z, Roscioli E, Murphy J, Ou J, Bassiouni A, Wormald P J, et al. Staphylococcus aureus impairs the airway epithelial barrier in vitro. Int Forum Allergy Rhinol. 2015;5:551-6.
24. Seop Kim C, Jeon S-Y, Min Y-G, Rhyoo C, Kim J-W, Bock Yun J, et al. Effects of β-Toxin of Staphylococcus aureus on Ciliary Activity of Nasal Epithelial Cells. Laryngoscope. 2000;110:2085-8.
25. Eiffler I, Behnke J, Ziesemer S, Muller C, Hildebrandt J-P. Staphylococcus aureus α-toxin-mediated cation entry depolarizes membrane potential and activates p38 MAP kinase in airway epithelial cells. Am J Physiol Lung Cell Mol Physiol. 2016;311:L676-85.
26. Hermann I, Rath S, Ziesemer S, Volksdorf T, Dress R J, Gutjahr M, et al. Staphylococcus aureus hemolysin A disrupts cell-matrix adhesions in human airway epithelial cells. Am J Respir Cell Mol Biol. 2015;52:14-24.
27. Yin J, Yu F-SX. Rho kinases regulate corneal epithelial wound healing. Am J Physiol Cell Physiol. 2008;295:378-87.
28. Elamin E, Masclee A, Dekker J, Jonkers D. Ethanol disrupts intestinal epithelial tight junction integrity through intracellular calcium-mediated Rho/ROCK activation. Am J Physiol Gastrointest Liver Physiol. 2014;306:G677-85.
29. Crosby L M, Waters C M. Epithelial repair mechanisms in the lung. Am J Physiol Lung Cell Mol Physiol. 2010;298: L715-31.
30. Sun C C, Chiu H T, Lin Y F, Lee K Y, Pang JHS. Y-27632, a ROCK inhibitor, promoted limbal epithelial cell proliferation and corneal wound healing. PLoS One. 2015;10:1-18.
31. Meltzer E O, Hamilos D L, Hadley J A, Lanza D C, Marple B F, Nicklas R A, et al. Rhinosinusitis: Establishing definitions for clinical research and patient care. J Allergy Clin Immunol. 2004;114:5155-212.
32. Ruffin M, Bilodeau C, Mailĺe É, LaFayette SL, McKay G A, Trinh N T N, et al. Quorum-sensing inhibition abrogates the deleterious impact of Pseudomonas aeruginosa on airway epithelial repair. FASEB J. 2016;30:3011-25.
33. Trinh N T, Bilodeau C, Mailĺe É, Ruffin M, Quintal M, Desrosiers M, et al. Deleterious impact of Pseudomonas aeruginosa on cystic fibrosis transmembrane conductance regulator function and rescue in airway epithelial cells. Eur Respir J. 2015;45:1590-602.
34. Trinh N, Bardou O, Privé A, Mailĺe E, Adam D, Lingée S, Ferraro P, Desrosiers M, Coraux C B E. Improvement of defective cystic fibrosis airway epithelial wound repair after CFTR rescue. Eur Respir J. 2012;40:1390-400.
35. Bilodeau C, Bardou O, Maillé É, Berthiaume Y, Brochiero E. Deleterious impact of hyperglycemia on cystic fibrosis airway ion transport and epithelial repair. J Cyst Fibros. 2016;15:43-51.
36. MailléE, Trinh N T N, Privé A, Bilodeau C, Bissonnette É, Grandvaux N, et al. Regulation of normal and cystic fibrosis airway epithelial repair processes by TNF-α after injury. Am J Physiol Lung Cell Mol Physiol. 2011;301:L945-55.
37. Trinh N T N, Prive A, Maille E, Noel J, Brochiero E. EGF and K+ channel activity control normal and cystic fibrosis bronchial epithelia repair. Am J Physiol Lung Cell Mol Physiol. 2008;295:L866-80.
38. Choi S, Camp S M, Dan A, Garcia J G, Dudek S M, Leckband D E. A genetic variant of cortactin linked to acute lung injury impair lamellipodia dynamics and endothelial wound healing. Am J Physiol Lung Cell Mol Physiol. 2015;309:L983-94.
39. Steffen A, Rottner K, Ehinger J, Innocenti M, Scita G, Wehland J, et al. Sra-1 and Nap1 link Rac to actin assembly driving lamellipodia formation. EMBO J. 2004;23:749-59.
40. Zhang N, van Crombruggen K, Gevaert E, Bachert C. Barrier function of the nasal mucosa in health and type-2 biased airway diseases. Allergy. 2016;71:295-307.
41. De Greve G, Hellings P W, Fokkens W J, Pugin B, Steelant B, Seys S F. Endotype-driven treatment in chronic upper airways diseases. Clin Trans Allergy. 2017;7-22.
42. Stevens W W, Lee R J, Schleimer R P, Cohen N A. Chronic rhinosinusitis pathogenesis. J Allergy Clin Immunol. 2015;136:1142-53.
43. Schleimer R P. Immunopathogenesis of chronic rhinosinusitis and nasal polyps. Annu Rev Pathol Mech Dis. 2017;12:331-57.
44. Duan C, Li C W, Zhao L, Subramaniam S, Yu X M, Li Y Y, et al. Differential expression patterns of egf, egfr, and erbb4 in nasal polyp epithelium. PLoS One. 2016;11:1-14.
45. Desai L P. RhoA and Rac1 are both required for efficient wound closure of airway epithelial cells. Am J Physiol Lung Cell Mol Physiol. 2004;287:L1134-44. DOI: 10.1152/ajplung.00022.2004.
46. de Bentzmann S, Polette M, Zahm J, Hinnrasky J, Kileztky C, Bajolet O, et al. Pseudomonas Aeruginosa Virulence Factors Delay Airway Epithelial Wound Repair by Altering the Actin Cytoskeleton and Inducing Overactivation of Epithelial Matrix Metalloproteinase-2. Lab Invest. 2000;80:209-19.
47. Menko A S, Bleaken B M, Walker J L. Regional-specific alterations in cell-cell junctions, cytoskeletal networks and myosin-mediated mechanical cues coordinate collectivity of movement of epithelial cells in response to injury. Exp Cell Res. 2014;322:133-48.
48. Horani A, Nath A, Wasserman M G, Huang T, Brody S L. Rho-associated protein kinase inhibition enhances airway epithelial basal-cell proliferation and lentivirus transduction. Am J Respir Cell Mol Biol. 2013;49:341-7.
49. Moore M, Marroquin B A, Gugliotta W, Tse R, White S R. Rho Kinase Inhibition Initiates Apoptosis in Human Airway Epithelial Cells. Am J Respir Cell Mol Biol. 2004;30:379-87.
50. Zuo W, Zhang T, Wu D Z, Guan S P, Liew A-A, Yamamoto Y, et al. p63+Krt5+distal airway stem cells are essential for lung regeneration. Nature. 2014;517:616-20.
51. Arason A J, Jonsdottir H R, Halldorsson S, Benediktsdottir B E, Bergthorsson J T, Ingthorsson S, et al. DeltaNp63 has a role in maintaining epithelial integrity in airway epithelium. PLoS One. 2014;9:e88683.
52. Crosby L M, Waters C M. Epithelial repair mechanisms in the lung. Am J Physiol Lung Cell Mol

Physiol. 2010;298:L715-31.
53. Marano R J, Wallace H J, Wijeratne D, Fear M W, Wong H S, O'Handley R. Secreted biofilm factors adversely affect cellular wound healing responses in vitro. Sci Rep. 2015;5:13296.
54. Brothers K M, Stella N A, Hunt K M, Romanowski E G, Liu X, Klarlund J K, et al. Putting on the brakes: Bacterial impediment of wound healing. Sci Rep. 2015;5:14003.
55. Wise S K, Den Beste K A, Hoddeson E K, Parkos C A, Nusrat A. Sinonasal epithelial wound resealing in an in vitro model: Inhibition of wound closure with IL-4 exposure. Int Forum Allergy Rhinol. 2013;3:439-49.
56. Hilliard J J, Datta V, Tkaczyk C, Hamilton M, Sadowska A, Jones-Nelson O, et al. Anti-alpha-toxin monoclonal antibody and antibiotic combination therapy improves disease outcome and accelerates healing in a Staphylococcus aureus dermonecrosis model. Antimicrob Agents Chemother. 2015;59:299-309.
57. Ljubimov A V., Saghizadeh M. Progress in corneal wound healing. Prog Retin Eye Res. 2015;49:17-45.
58. Mfuna-Endam L, Zhang Y, Desrosiers M Y. Genetics of rhinosinusitis. Curr Allergy Asthma Rep. 2011;11:236-46.
59. Iorio V, Troughton L D, Hamill K J. Laminins: Roles and Utility in Wound Repair. Adv Wound Care.

2015;4:250-63.
60. Purkey M T, Li J, Mentch F, Grant S F, Desrosiers M, Hakonarson H, et al. Genetic variation in genes encoding airway epithelial potassium channels is associated with chronic rhinosinusitis in a pediatric population. PLoS One. 2014;9:e89329.
61. Girault A, Chebli J, Privé A, Trinh N T N, Maillé E, Grygorczyk R, et al. Complementary roles of KCa3.1 channels and β-integrin during alveolar epithelial repair. Respir Res. 2015;16:100.
62. Girault A, Brochiero E. Evidence of K+channel function in epithelial cell migration, proliferation, and repair. Am J Physiol Cell Physiol. 2014;306:C307-19.
63. Cormier C, Mfuna Endam L, Filali-Mouhim A, Boisvert P, Boulet L-P, Boulay M-E, et al. A pooling-based genomewide association study identifies genetic variants associated with Staphylococcus aureus colonization in chronic rhinosinusitis patients. Int Forum Allergy Rhinol. 2014;4:207-15.
64. Saint-Criq V, Villeret B, Bastaert F, Kheir S Hatton A, Cazes A, et al. Pseudomonas aeruginosa LasB protease impairs innate immunity in mice and humans by targeting a lung epithelial cystic fibrosis transmembrane regulator-IL-6-antimicrobial-repair pathway. Thorax. 2017;Epub Ahead.
65. Popoff M R. Bacterial factors exploit eukaryotic Rho GTPase signaling cascades to promote invasion and proliferation within their host. Small GTPases. 2014;5:e28209.
66. Boyer L, Doye A, Rolando M, Flatau G, Munro P, Gougon P et al. Induction of transient macroapertures in endothelial cells through RhoA inhibition by Staphylococcus aureus factors. J Cell Biol. 2006;173:809-19.
67. Dach K, Zovko J, Hogardt M, Koch I, van Erp K, Heesemann J, et al. Bacterial toxins induce sustained mRNA expression of the silencing transcription factor klf2via inactivation of RhoA and Rhophilin 1. Infect Immun. 2009;77:5583-92.
68. Soong G, Martin F J, Chun J, Cohen T S, Ahn D S, Prince A. Staphylococcus aureus protein A mediates invasion across airway epithelial cells through activation of RhoA GTPase signaling and proteolytic activity. J Biol Chem. 2011;286:35891-8.
69. Narimatsu T, Ozawa Y, Miyake S, Kubota S, Hirasawa M, Nagai N, et al. Disruption of cell-cell junctions and induction of pathological cytokines in the retinal pigment epithelium of light-exposed mice. Invest Ophthalmol Vis Sci. 2013;54:4555-62.

What is claimed is:

1. A method for promoting sinonasal epithelial cell repair in a subject in need thereof comprising administering to said subject an effective amount of a Rho/ROCK signaling pathway inhibitor.

2. The method of claim 1, wherein the Rho/ROCK signaling pathway inhibitor is a Rho kinase (ROCK) inhibitor.

3. The method of claim 2, wherein the ROCK inhibitor is an antibody or an antigen-binding fragment thereof that binds to ROCK1 and/or ROCK2 and blocks its activity.

4. The method of claim 2, wherein the ROCK inhibitor is an antisense compound specifically hybridizes with a nucleic acid encoding human ROCK1 and/or human ROCK2.

5. The method of claim 2, wherein the ROCK inhibitor is Fasudil (HA-1077), Hydroxyfasudil, Netarsudil, Ripasudil, RKI-1447, GSK429286A, GSK180736A, GSK269962A, Thiazovivin, AT13148, H1152, Glycyl-H1152, TC-S 7001, AS-1892802, HA-1100, OXA-06, SB-772077B, SR-3677, KD025 (SLx-2119), Y-30141, Y-39983, ZINC00881524 or Y-27632, or a pharmaceutically acceptable salt thereof.

6. The method of claim 5, wherein the ROCK inhibitor is Y-27632 or a pharmaceutically acceptable salt thereof.

7. The method of claim 6, wherein the ROCK inhibitor is a dihydrochloride salt of Y-27632.

8. The method of claim 1, wherein the subject suffers from rhinosinusitis.

9. The method of claim 8, wherein the subject suffers from chronic rhinosinusitis (CRS).

10. The method of claim 9, wherein the subject suffers from CRS with nasal polyps (CRSwNP).

11. The method of claim 1, wherein the subject underwent endoscopic sinus surgery (ESS) prior to said administering.

12. The method of claim 8, wherein the rhinosinusitis is associated with a bacterial infection.

13. The method of claim 12, wherein the bacterial infection comprises Staphylococcus aureus infection.

14. The method of claim 1, wherein the Rho/ROCK signaling pathway inhibitor is formulated in a pharmaceutical composition with at least one pharmaceutically acceptable carrier or excipient.

15. The method of claim 1, wherein the Rho/ROCK signaling pathway inhibitor is administered intranasally.

16. The method of claim 15, wherein the Rho/ROCK signaling pathway inhibitor is coated on a nasal or sinus implant.

17. The method of claim 1, wherein the Rho/ROCK signaling pathway inhibitor is administered in combination with at least one additional active agent.

18. The method of claim 17, wherein the at least one additional active agent comprises a steroid and/or an antibiotic.
